(19) United States
(12) Patent Application Publication
(10) Pub. No.: US 2017/0182208 A1
(43) Pub. Date: Jun. 29, 2017

(54) ABSORBABLE FIBRIN MICROTHREAD SUTURES FOR REDUCED INFLAMMATION AND SCARRING IN TISSUE LIGATION

(71) Applicant: VITA THREADS,LLC, Worcester, MA (US)

(72) Inventors: George PINS, Worcester, MA (US); Glenn GAUDETTE, Worcester, MA (US); Adam COLLETTE, Worcester, MA (US); William EDELMAN, Worcester, MA (US)

(21) Appl. No.: 15/129,709

(22) PCT Filed: Mar. 27, 2015
(86) PCT No.: PCT/US2015/023124

§ 371 Date: Sep. 27, 2016

Related U.S. Application Data
(60) Provisional application No. 61/971,752, filed on Mar. 28, 2014.

(51) Int. Cl. A61L 17/08; D01F 4/00; D01D 5/00; A61L 17/00

(52) U.S. Cl. CPC A61L 17/08; A61L 17/005; D01F 4/00; D01D 5/00; D10B 2509/04; D10B 2401/12; D10B 2211/20

(57) ABSTRACT

In part, the invention described herein relates generally to sutures and uses thereof in surgical procedures, including fibrin microthread sutures for surgical procedures that provide one or more of lower inflammation, reduced fibrosis, reduced scarring, and fast absorption in the host tissue.

### RELATED APPLICATIONS

This application claims the benefit of U.S. Provisional Patent Application No. 61/971,752, filed Mar. 28, 2014, which is hereby incorporated by reference in its entirety.

### BACKGROUND

Embodiments described herein relate generally to sutures for surgical procedures, and in particular to fibrin microthread sutures for surgical procedures that provide lower inflammation, reduced scarring, and fast absorption in the host tissue.

Multicellular organisms including mammals are made up of tissues which are organized aggregates of specialized group of cells. When tissues become damaged, for example, from an injury, or a surgical procedure, physiological events take place to close and repair the damaged site (e.g., an open wound, a gash, a surgical incision, etc.), and allow successful repair and regeneration of the tissue. These physiological events include an inflammatory response in which neutrophils, eosinophils, macrophages, lymphocytes, fibrocytes, and other cells involved in the inflammatory response migrate to the damage site to promote blood clotting and remove bacteria, debris and damaged tissue. Later, circulating cells migrate to the wound site and differentiate into myofibroblasts. The differentiated cells begin to deposit new extracellular matrix, which includes a complex assemblage of proteins, carbohydrates, and collagen, that provide support and anchorage for the cells. Depending on the method used to close and repair the wound, excessive connective tissue and collagen can be deposited at the damage site. This is referred to as fibrosis and if sufficiently extensive can lead to scar formation, which is undesirable in most procedures and can be particularly undesirable in topical or cosmetic surgical procedures.

Sutures are often used to ligate or close an open wound as an augmentation to the native response, for example, an incision formed during a surgical procedure. Sutures can be absorbable or non-absorbable. Non-absorbable sutures such as, polypropylene, polyester, polyvinylidene fluoride, nylon, stainless steel or silk sutures are not biodegradable and may have to be physically removed from the wound site or elsewise allowed to remain in place permanently. Both their presence and the process of their removal can lead to substantial inflammation at the ligature site.

In contrast, absorbable sutures are biodegradable and can be absorbed or broken down by the host tissue through a variety of mechanisms. Such absorbable sutures can include natural sutures such as, for example, surgical gut, fast-absorbing gut, cat gut, collagen, or synthetic absorbable sutures such as, for example, polyglycolic acid, polylactic acid, polydioxanone, poliglecaprone (MONOCRYL®), polyglactin (VICRYL®), and caprolactone sutures. State of the art conventional absorbable sutures, however, can cause inflammation and scarring at the ligature site due to their decomposition byproducts and the cellular functions necessary in their breakdown. Furthermore, conventional absorbable sutures can take a substantial amount of time to absorb in the host tissue. For example, catgut sutures are absorbed in the host tissue in about 70 days, polyglycolic acid in about 60-90 days, MONOCRYL® in about 60-90 days, polydioxanone in about 180-210 days, and VICRYL® in about 56-70 days. These long absorption times can further promote phenomena including inflammation, granuloma formation, suture extrusion, and scarring at the ligature site.

Thus there is a need for new sutures that provide lower inflammation, reduced fibrosis, reduced scarring, and fast absorption in host tissue.

### SUMMARY

Embodiments described herein relate generally to sutures for surgical procedures, and in particular to fibrin microthread sutures for surgical procedures that provide one or more of lower inflammation, reduced fibrosis, reduced scarring, and fast absorption in the host tissue. In some embodiments, a suture for ligating incision wounds includes fibrin microthreads. The fibrin microthread may be associated with one or more of a substrate or a braided yarn or other hierarchically organized rope, a woven or non-woven mesh, a surgical needle, a surgical pin, a surgical screw, a surgical plate, a physiologically acceptable patch, a dressing, a bandage, or a natural or mechanical valve. In certain embodiments, the fibrin microthread includes an additional therapeutic agent.

In some embodiments, the fibrin microthreads are formed by combining a first volume of fibrinogen with a second volume of a molecule capable of forming fibrin from fibrinogen to form a mixture. The mixture is transferred to a lumen containing device and a distal end of the lumen containing device is disposed into an aqueous bath. The mixture is extruded from the distal end of the lumen containing device while moving the distal end through the aqueous bath. The fibrin microthreads are substantially formed in the aqueous bath and are then removed from the aqueous bath. In some embodiments, the fibrin microthreads substantially absorb within a host tissue at the ligature site in about 3 to about 28 days. In an embodiment, the fibrin microthreads substantially absorb within a host tissue at the ligature site in about 4 to about 10 days. In an embodiment, the fibrin microthreads substantially absorb within a host tissue at the ligature site in about 7 to about 14 days. In an embodiment, the fibrin microthreads substantially absorb within a host tissue at the ligature site in about 14 to about 28 days. In some embodiments, sutures with fibrin microthreads lead to less collagen deposition than a conventional suture, including, for example, polyglactin (VICRYL®). Other illustrative conventional sutures include, but are not limited to, surgical gut, cat gut, collagen, or synthetic absorbable sutures such as, for example, polyglycolic acid, polylactic acid, polydioxanone, poliglecaprone (MONOCRYL®), and caprolactone sutures. In some embodiments, the fibrin microthreads have a histopathological score of collagen deposition at the ligature site of less than about 1.3. In some embodiments, sutures with fibrin microthreads lead to less overall inflammation at the ligature site than a conventional suture, including, for example, polyglactin (VICRYL®). In some embodiments, the fibrin microthreads have a histopathological score of overall inflammation at the ligature site of less than about 1.3. In some embodiments, sutures with fibrin microthreads are well resorbed at the ligature site and display superior resorption properties than a conventional suture, including, for example, polyglactin (VICRYL®). In some embodiments, the fibrin microthreads have a histopathological score of extent of resorption of greater than 1, and even as high as about 3.0. In some embodiments, sutures with fibrin microthreads lead to less overall cellular infiltration or proliferation at the ligature site than a conventional suture, including, for example, polyglactin (VICRYL®).

### BRIEF DESCRIPTION OF DRAWINGS

FIG. 1A shows dry fibrin microthread disposed in a container. FIG. 1B shows the fibrin microthread of FIG. 1A disposed in a lactated ringer's solution to hydrate the fibrin microthread.

FIG. 2A shows a 3 cm full thickness dorsal skin incision in a Sprague Dawley rat. FIG. 2B shows an incision being performed on the dorsal skin of the rat by a surgical scalpel.

FIGS. 3A-D show the incision wound shown in FIG. 2B being ligated by the fibrin microthread of FIG. 1B using a running intradermal stitch pattern.

FIGS. 4A-D show post-ligation images of the surgical wounds using a running intradermal stitch pattern.

FIG. 5A shows incision wounds ligated with the fibrin microthread of FIG. 1B using a running intradermal stitch pattern one day after ligation. FIG. 5B shows incision wounds ligated with a VICRYL® suture using a running intradermal stitch pattern one day after ligation.

FIG. 6 shows the incision wound ligated with the fibrin microthread and the incision wounds ligated with the VICRYL® suture 1 week after ligation in both cases using a running intradermal stitch pattern.

FIG. 7 panel A, B and C show histopathological images detailing the acute histology of incision wounds ligated using the fibrin microthread at 1 day, 3 days and 7 days post-ligation. Panel D, E and F show histopathological images detailing the acute histology of incision wounds ligated using VICRYL® suture at 1 day, 3 days and 7 days post-ligation. Panel G, H and I show histopathological images detailing the acute histology of incision wounds ligated using surgical gut suture at 1 day, 3 days and 7 days post-ligation.

FIG. 8 panel A, B and C show histopathological images detailing the acute histology of incision wounds ligated using the fibrin microthread at 1 day, 3 days and 7 days post-ligation. Panel D, E and F show histopathological images detailing the acute histology of incision wounds ligated using VICRYL® suture at 1 day, 3 days and 7 day post-ligation. Panel G, H and I show histopathological images detailing the acute histology of incision wounds ligated using surgical gut suture at 1 day, 3 days and 7 days post-ligation.

FIG. 9 panel A, B and C show histopathological images detailing early chronic histology of incision wounds ligated using the fibrin microthread at 14 days, 28 days, and 57 days post-ligation. Panel D, E and F show histopathological images detailing early chronic histology of incision wounds ligated using VICRYL® suture at 14 days, 28 days and 57 days post-ligation. Panel G shows a histopathological image detailing the early chronic histology of an incision wound ligated using surgical gut suture at 14 days post-ligation.

FIG. 10 panel A, B and C show histopathological images detailing early chronic histology of incision wounds ligated using the fibrin microthread at 14 days, 28 days, and 57 days post-ligation. Panel D, E and F show histopathological images detailing early chronic histology of incision wounds ligated using VICRYL® suture at 14 days, 28 days and 57 days post-ligation. Panel G shows a histopathological image detailing the early chronic histology of an incision wound ligated using surgical gut suture at 14 days post-ligation.

FIG. 11 is a plot of resorption scores of fibrin microthread and VICRYL® sutures in the host tissue.

FIG. 12 is a plot of inflammation scores of inflammation at wound sites site ligated using fibrin microthread and VICRYL® sutures.

FIG. 13 is a plot of a collagen deposition scores at wound sites ligated using fibrin microthread and VICRYL® sutures.

FIG. 14A shows a 3 cm full thickness dorsal skin incision in a Sprague Dawley rat closed using a fibrin microthread suture (top) or a 6-0 fast-absorbing gut suture (bottom) using a simple interrupted transcutaneous suture pattern immediately post-operatively. FIG. 14B shows close-up detail on fibrin microthread suture-closed incisions of FIG. 14A. FIG. 14C shows close-up detail on fast-absorbing gut suture-closed incisions of FIG. 14A.

FIG. 15A shows a pair of 3 cm full thickness dorsal incisions in a Sprague Dawley rat 4 days post-operatively having been closed using fibrin microthread suture using a simple interrupted transcutaneous suture pattern demonstrating continued presence of fibrin microthread suture. FIG. 15B shows a pair of 3 cm full thickness dorsal incisions in a Sprague Dawley rat 4 days post-operatively having been closed using fibrin microthread suture using a simple interrupted transcutaneous suture pattern demonstrating initial sloughing off of fibrin microthread suture. FIG. 15C shows a pair of 3 cm full thickness dorsal incisions in a Sprague Dawley rat 4 days post-operatively having been closed using 6-0 fast absorbing gut suture using a simple interrupted transcutaneous suture pattern demonstrating both continued presence and sloughing off of fast absorbing gut suture.

FIG. 16 shows the dorsal skin of a rat 28 days post-operatively after a series of four 3 cm incisions were generated (incision endpoints denoted by dots placed with marker pen). Closures were performed using a simple interrupted transcutaneous suture pattern and fibrin microthread sutures (2 closures at right) or 6-0 fast-absorbing gut suture (2 closures at left).

FIG. 17A and FIG. 17B show a representative example 3 cm×1 cm skin tag harvested from a healed incision wound in native and inverted orientations prior to single-pull-to-failure tensile testing. FIG. 17C shows the results of a completed tensile test with a clean skin break and remnant stumps located in either test grip.

FIG. 18A and FIG. 18B show a table describing the mean of all recorded maximum failure loads for repaired skin incision tags as illustrated in FIG. 17A, FIG. 17B, and FIG. 17C as seen in repairs performed using a simple interrupted transcutaneous suture pattern with either 10-ply fibrin microthread suture, 6-0 nylon suture, or 6-0 fast-absorbing gut suture. Minimum sample number per sample suture type per timepoint was n=6

FIGS. 19A-19D show the production of fibrin microthreads using a three axis electromechanical extrusion head. FIGS. 19A and 19B show automation of thread extrusion via a three axis thread extruder allows for consistent production of fibrin microthreads. FIGS. 19C and 19D show that microthreads produced in accordance with methods of the invention have consistent diameter (as measured using a yarn micrometer) and failure load (as measured via uniaxial pull to failure).

FIGS. 20A-20C show the production process for multi-filament and mono-filament fibrin microthread sutures. FIG. 20A shows production differences between multi-filament and mono-filament fibrin microthread sutures. FIG. 20B shows a scanning electron microscope (SEM) micrograph of multi-filament suture cross section. FIG. 20C shows a SEM micrograph of mono-filament suture cross-section. (Scale bars 100 μm).

FIGS. 21A-21C show fibrin microthreads of the invention. FIG. 21A shows fibrin microthread sutures produced in six different form-factors and sizes. As shown, the sutures are associated with different needle sizes. Sutures can also be associated with consistently sized needles. FIG. 21B show the schematics of fibrin microthread sutures implanted into a rat dorsal skin closure model. FIG. 21C show the schematics of testing fibrin microthread sutures via biological assessment and wound mechanics.

FIGS. 22A-22D show mechanical characterization of fibrin microthreads. FIG. 22A shows mechanical testing of fibrin microthreads. FIG. 22B shows that fibrin microthread tensile strength increases with thrombin concentration. FIG. 22C shows that fibrin microthread strength increases with addition of CaCl₂. FIG. 22D shows that the addition of a stretching protocol and drying step increases microthread tensile strength. (*indicates p<0.05)

### DETAILED DESCRIPTION

The present invention is based, in part, on the discovery that fibrin microthreads provide desirable wound closure characteristics that improve upon conventional suture technology. Embodiments described herein relate generally to sutures for surgical procedures, and in particular to fibrin microthread sutures for surgical procedures that provide, for example, lower inflammation, reduced fibrosis, reduced scarring, and fast and more extensive absorption in the host tissue. Accordingly, the present invention provides, in part, various compositions and methods for wound closure with a fibrin microthread, optionally a monofilament fibrin microthread, and/or uses of a fibrin microthread, optionally a monofilament fibrin microthread in wound closure and/or in the manufacture of a medicament for wound closure. As described herein, the present methods and uses can include a variety of surgical techniques, including plastic surgery.

Absorbable sutures are biodegradable and can get absorbed or broken down by the host tissue. Such absorbable sutures can include natural sutures such as, for example, surgical gut, cat gut, collagen, or synthetic absorbable sutures such as, for example, polyglycolic acid, polylactic acid, polydioxanone, poliglecaprone (MONOCRYL®), polyglactin (VICRYL®), and caprolactone sutures. State of the art conventional absorbable sutures, however, can cause substantial inflammation and scarring at the ligature site.

Inflammation and scarring as a byproduct of fibrosis are a particular concern in topical wound ligatures and ligatures performed during cosmetic procedures. For example, approximately 1.7 million plastic surgery procedures are performed every year in the United States alone. It is desirable in plastic surgery procedures and other high sensitivity or high visibility tissue ligation applications such as sensitive facial tissue surgical procedures, that the ligation sutures produce minimal inflammation and scarring, and absorb fast in the host tissue. Additional surgeries such as peripheral nerve surgery and detached retina surgery would also benefit substantially from a minimally inflammatory absorbable suture.

Inflammation can be measured in various ways. Chronic inflammation can be measured using the erythrocyte sedimentation rate (ESR) test. The ESR test is a non-specific test in which a blood sample from a host (e.g., a patient) is disposed in a container, for example, a vial or a tube and maintained in the vial or tube for about an hour or more. The amount of red blood cells that settle to the bottom of the container in 1 hour is used as a non-specific marker in determining the level of inflammation in the host. Enzyme linked immunosorbent assay (ELISA) can also be used to determine the concentration of inflammation biomarkers (e.g., cytokines, C-reactive protein, interleukin-6, or any other inflammation biomarkers) in a blood or plasma sample and determine inflammation in the host. Coulter counters can also be used to count the number of white blood cells, neutrophils, eosinophils, macrophages, lymphocytes, leukocytes, granulocytes, or any other immune cell included in the host immune response that can cause inflammation.

Inflammation can also be assessed using histological techniques, as are known in the arts (see e.g., Ross and Pawlina (2006). Histology: A Text and Atlas. Hagerstown, MD: Lippincott Williams and Wilkins, the contents of which are hereby incorporated by reference herein in their entirety). For example, hematoxylin and eosin (H&E or HE) may be used with, for example, light microscopy. Hematoxylin, a basic dye, stains nuclei blue because of its affinity for nucleic acids in the cell nucleus. Eosin, an acidic dye, stains the cytoplasm pink. Trichrome is another common staining method that includes three colored dyes that can be formulated to stain erythrocytes orange (or yellow), muscle red and collagen blue. Red dyes that can be used in the trichrome stain include without limitation acid fuchsin, xylidine ponceau, chromotrope 2R, biebrich scarlet, ponceau 6R, and phloxine. Blue dyes that can be used in the trichrome stain include without limitation aniline blue, methyl blue, and water blue. Yellow dyes that can be used in the trichrome stain include without limitation picric acid, orange G, martius yellow, tartrazine, and milling yellow. Uranyl acetate and lead citrate may be used to impart contrast to tissue in, for example, the electron microscope. Furthermore, any one of the following illustrative stains may be used: Toluidine blue, Masson's trichrome stain, Weigert's elastic stain, Heidenhain's AZAN trichrome stain, silver stain, Wright's stain, Orcein stain, periodic acid-Schiff (PAS) stain, any other suitable stain or combination thereof. Such stains are interpreted as common in histological analysis (including as disclosed by Ross and Pawlina (2006). Histology: A Text and Atlas. Hagerstown, Md.: Lippincott Williams and Wilkins).

The level of acute inflammation, for example, inflammation of the host tissue due to an incision wound, a suture, or a physical injury, can also be measured using histopathological scoring of histology samples, for example, histology samples stained with any of the stains or dyes described herein. The inflammation in the histopathological stains is quantified using a semi-quantitative scale called the histological activity index (HAI). The scale ranges from a minimum inflammation activity score of 0 to a maximum score of 3, where:

0=No inflammatory activity/None (no infiltration of the epithelium by neutrophils);

1=Mildly active/Trace (Neutrophil infiltration of <50% of sampled crypts or cross sections, no ulcers or erosions);

2=Moderately active/Apparent (Neutrophil infiltration of 50% of sampled crypts or cross sections, no ulcers or erosions);

3=severely active/Prominent (Erosion or ulceration, irrespective of other features).

In some embodiments, the fibrin microthreads described herein (which may include, without limitation monofilament forms) can be used in surgical procedures such as, for example, ligating open wounds or incisions such that the inflammation elicited in the host tissue or proximal to the suture site due to implantation of the fibrin microthreads (by way of non-limiting example, when used in plastic surgery) is substantially less than inflammation caused by conventional sutures. In some embodiments, the fibrin microthreads can have a histopathological score of overall inflammation in the host tissue of less than about 1.5 such as, for example, about 1.4, or about 1.3, or about 1.2, or about 1.1, or about 1.0, or about 0.9, or about 0.8, or about 0.7, or about 0.6, or about 0.5, or about 0.4, or about 0.3, or about 0.2, or about 0.1, or even about 0. In a specific embodiment, the fibrin microthreads have a histopathological score of overall inflammation at the ligature site of less than about 1.3.

In various embodiments, the present fibrin microthreads reduce inflammation at the suture site as characterized by a reduction in one or more of edema, erythema, tenderness, induration, discharge, and nodule formation relative to suturing with conventional materials. In some embodiments, the present fibrin microthreads prevent or reduce cellular infiltration or proliferation at the host tissue or proximal to the fibrin microthread suture.

The amount of fibrosis or scarring (e.g., collagen deposition) in a host tissue or proximal to the suture site, for example, after healing of an incision wound, a suture, or any other physical injury healing can also be determined using visual methods. Examples of visual methods used for measuring the extent of scarring include: (a) the Vancouver scar scale (VSS) that ranges from 0-13 and quantifies scars on the basis of vascularity, height/thickness, pliability, and pigmentation; (b) the visual analog scale (VAS) that ranges from 0 (excellent) to 100 (poor) and quantifies scars on the basis of vascularity, pigmentation, acceptability, observer comfort plus contour and summing the individual scores; (c) the patient and observer scale which ranges from 5-50 and quantifies scars on the basis of VSS plus surface area; patient assessments of pain, itching, color, stiffness, thickness, relief; (d) the Manchester scar scale which ranges from 5 (best) to 18 (worse) and quantifies scars on the basis of VAS plus scar color, skin texture, relationship to surrounding skin, texture, margins, size, multiplicity; and (e) the Stony Brook scar scale which ranges from 0 (worst) to 5 (best) and quantifies scars on the basis of VAS plus width, height, color, presence of suture/staple marks. Fibrosis or scarring can also be quantified in terms of collagen deposition by performing histology analysis, for example, analysis of histology samples stained with any suitable dyes or stains described herein (including as disclosed by Ross and Pawlina (2006). Histology: A Text and Atlas. Hagerstown, Md.: Lippincott Williams and Wilkins) and using the HAI scale described herein.

In some embodiments, the fibrin microthreads can elicit substantially reduced scarring in the host tissue or proximal to the suture site relative to conventional sutures (by way of non-limiting example, when used in plastic surgery). In various embodiments, the present sutures provide for reduced scarring assessed by one or more of the VSS (e.g. lower scores, such as about 0, or about 1,or about 2, or about 3), the VAS (e.g. lower scores, such as about 0, or less than about 5, or less than about 10, or less than about 15, or less than about 20, or less than about 25), the patient and observer scale (e.g., lower scores, such as about 5, or about 6, or about 7, or about 8, or about 9, or about 10), Manchester scar scale (e.g. lower scores, such as about 5, or about, 6, or about 7, or about 8, or about 9), and the Stony Brook scar scale (e.g. higher scores, such as about 3, or about 4, or about 5). In some embodiments, the fibrin microthreads can elicit substantially reduced collagen deposition in the host tissue or proximal to the suture site relative to conventional sutures and thereby, result in reduced scarring at the wound site. In some embodiments, the fibrin microthreads can have a histopathological score of collagen deposition of less than about 1.5 (e.g., about 7 days, or 14 days after implantation in a host tissue), for example, about 1.4, about 1.3 about 1.2, about 1.1, about 1, about 0.5, or about 0 (e.g., about 7 days, or 14 days after implantation in a host tissue). In a specific embodiment, the fibrin microthreads have a histopathological score of collagen deposition at the ligature site of less than about 1.3 (e.g., about 7 days, or 14 days after implantation in a host tissue).

In some embodiments, the fibrin microthreads described herein provide fast and more extensive absorption in the host tissue (by way of non-limiting example, when used in plastic surgery). In some embodiments, the fibrin microthreads described herein can have an extent of resorption of greater than about 1 (e.g., about 7, or about 14 days after implantation in a host tissue), for example about 1.1, 1.3, 1.5, 1.7, 1.9, 2.0.2.2, 2.4, 2.6, 2.8 or about 3 (e.g., about 7, or about 14 days after implantation in a host tissue), inclusive of all ranges therebetween. In a specific embodiment, the fibrin microthreads have a histopathological score of extent of resorption of less than about 3.0 (e.g., about 7, or about 14, days after implantation in a host tissue).

The fibrin microthreads can also absorb substantially faster in the host tissue relative to conventional sutures. In some embodiments, the fibrin microthreads described herein can absorb in the host tissue in a substantially shorter amount of time than conventional sutures. In some embodiments, fibrin microthread can be absorbed in the host tissue in the range of about 3 to about 28 days, about 4 to about 10 days, about 7 to about 14 days, about 14 to about 28 days, or about 6 days to about 16 days, for example, 7 days, 8 days, 9 days, 10 days, 11 days, 12 days, 13 days, 14 days, or about 15 days. In a specific embodiment, the fibrin microthreads substantially absorb within a host tissue at the ligature site in about 4 to about 10 days. In a specific embodiment, the fibrin microthreads substantially absorb within a host tissue at the ligature site in about 7 to about 14 days. In a specific embodiment, the fibrin microthreads substantially absorb within a host tissue at the ligature site in about 14 to about 28 days.

Embodiments of the fibrin microthreads described herein can be used for performing any desired surgical procedure or suture technique. In some embodiments, the fibrin microthreads can be used to perform plastic or cosmetic surgical procedures. For example, in some embodiments the fibrin microthreads can be used as sutures in a facial plastic surgery procedure including, but not limited to blepharoplasty, rhinoplasty, rhytidectomy, coronoplasty, chin augmentation, facial implants, ear surgery, hair implantation, cleft lip and cleft palate repair. In some embodiments, the fibrin microthreads can be used as sutures in a body plastic surgery procedure including but not limited to abdominoplasty, arm lift, thigh lift, breast reduction, breast augmentation, body contouring, C-section scar revision, hand surgery, liposuction, and any other cosmetic or plastic surgery procedure or combination thereof. In such embodiments, the fibrin microthreads can be used in place of conventional sutures such that there is substantially reduced inflammation and scarring of the host tissue after the surgical procedure. Furthermore, the fibrin microthreads can be absorbed into the host tissue much faster than conventional sutures, for example, within about 7 to about 14 days. Thus, use of fibrin microthreads in plastic and cosmetic surgery procedures can lead to faster recovery and substantially no visible signs of the incision wounds formed during the surgical procedure.

The fibrin microthreads described herein can be used to ligate or repair soft tissue such as, for example, simple squamous epithelia, stratified squamous epithelia, cuboidal epithelia, or columnar epithelia, connective tissue (e.g., loose connective tissue (also known as areolar connective tissue), fibrous connective tissue (e.g., tendons, which attach muscles to bone, and ligaments, which joint bones together at the joints), and muscle tissue (e.g., skeletal muscle, which is responsible for voluntary movements; smooth muscle, which is found in the walls of the digestive tract, bladder arteries and other internal organs; and cardiac muscle, which forms the contractile wall of the heart). The fibrin microthreads can be used to repair soft tissues in many different organ systems that fulfill a range of physiological functions in the body. These organ systems can include, but are not limited to, the muscular system, the genitourinary system, the gastroenterological system, the integumentary system, the circulatory system and the respiratory system. The fibrin microthreads are particularly useful for repairing connective tissue such as, for example, tendons and ligaments.

In some embodiments, the fibrin microthreads described herein can be used as sutures in any one of a cardiac surgery, skeletal muscle repair, congenital or incision hernia repair, abdominal surgery, laproscopic incision closure, organ prolapse surgery, gastrointestinal surgery, neurosurgery, severed limb reattachment surgery, open heart surgery, pulmonary surgery, hepatic surgery, renal surgery, ocular surgery, periodontal surgery, orthopedic surgery, and any other surgical procedure or combination thereof. In any such embodiments, the fibrin microthreads described herein can provide substantially reduced inflammation, little or no scarring, and fast and extensive adsorption into the host tissue within 7-14 days.

Embodiments of the fibrin microthread described herein can overcome the limitations of conventional surgical sutures. Embodiments of the fibrin microthread described herein provide several advantages over conventional suture including, for example: (1) substantially reduced inflammation relative to conventional sutures; (b) substantially reduced collagen deposition at the ligation site leading to little or no fibrosis or scarring; and (c) faster absorption in host tissue. Thus, embodiments of the fibrin microthreads described herein can be particularly beneficial for use as surgical sutures in high visibility and/or high sensitivity tissue ligation applications such as, for example, plastic surgery procedures. Methods of making embodiments of the fibrin microthreads described herein are described in U.S. Patent Publication No. 2011/0034388, entitled “Collagen and Fibrin Microthreads in a Discrete Thread Model of In Vitro ACL Scaffold Generation”, filed Mar. 15, 2007, the entire contents of which are hereby incorporated by reference. Embodiments of the fibrin microthreads described herein can also be seeded with cells, for example, stems cells (e.g., hematopoetic mesenchymal stem cells) to a ligation site. Methods of delivering fibrin microthreads to a ligation site for repairing or ameliorating damaged or defective tissue are described in U.S. Patent Publication No. 2011/0034867, entitled “Microthread Delivery System”, filed Feb. 10, 2011, the entire contents of which are hereby incorporated by reference.

In some embodiments, a suture for ligating incision wounds includes fibrin microthreads. The fibrin microthreads are formed by combining a first volume of fibrinogen with a second volume of a molecule capable of forming fibrin from fibrinogen to form a mixture. The mixture is transferred to a lumen containing device and a distal end of the lumen containing device is disposed in an aqueous bath. The mixture is extruded from the distal end of the lumen containing device while moving the distal end through the aqueous bath. The fibrin microthreads substantially form in the aqueous bath and are then removed from the aqueous bath. In some embodiments, the fibrin microtheads are mechanically stretched prior to or following removal from the aqueous bath.

As used herein, the term “about” and “approximately” generally mean plus or minus 10% of the value stated, for example about 250 μm would include 225 μm to 275 μm, about 1,000 μm would include 900 μm to 1,100 μm.

As used herein, the term “absorption” and “resorption” are used interchangeably to refer to the absorption of the sutures described herein into the host tissue in which the suture is implanted.

Embodiments of the fibrin microthreads described herein can be formed using a mechanical process. For example, in some embodiments, the fibrin microthreads can be formed by combining fibrinogen and a molecule capable of forming fibrin form the fibrinogen, for example, the enzyme thrombin, to form a mixture. The mixture can be transferred to a lumen containing device, for example, a tube or a conduit. A distal end of the lumen containing device can be disposed in an aqueous bath. The mixture can be extruded from the distal end of the lumen containing device, while moving the distal end of the lumen containing device through the aqueous bath. This deposits the mixture in the aqueous bath that is allowed to form into the fibrin microthread, for example, after incubating for a predetermined time.

Examples of apparatus, processes and methods that can be used for forming the fibrin microthreads described herein are described in U.S. Patent Publication No. 2011/0034388, the entire contents of which are hereby incorporated by reference. In illustrative embodiments, the lumen containing device can include a stabilized crosshead on a threaded rod with a crosshead speed of 4.25 mm/min through a blending applicator tip (Micromedics, Inc., St. Paul, Minn.). The blending applicators can be Luer locked to the two syringes through individual bores and mixed in a needle that is Luer locked to the tip. The fibrinogen and, for example, a thrombin solution, can be combined and extruded through polyethylene tubing (BD, Sparks, Md.) into an aqueous bath.

The rate of extrusion can vary according to the type of extrusion apparatus that is employed. The rate of extrusion can be expressed as a “rate ratio”, i.e., the ratio of flow velocity/plotter velocity, where flow velocity is the speed with which the fibrin solution emerges from the tubing and plotter velocity is the speed of the extrusion tubing through the aqueous bath. For example, a rate ratio of 2.0 describes extrusion parameters in which the solution flows out of the tubing twice as fast as the tubing tip moves through the aqueous bath. In various embodiments, the rate ratios for the apparatus described above can range, for example, from about 1.5 to about 6.0, e.g., about 1.6, about 1.8. about 2.0, about 2.5, about 3.0, about 3.5, about 4.0, about 4.5, about 5.0, or about 5.5.

The diameter of the tubing, i.e., the orifice from which the mixtures are extruded may also vary. For example, the diameter of the orifice has a diameter that can range from about 0.1 μm to about 1,000 μm, (e.g., about 1000 μm, about 500 μm, about 250 μm, about 200 μm, about 150 μm, about 100 μm, about 50 μm, about 40 μm, about 30 μm, about 20 μm, about 10 μm or about 1 μm, inclusive of all ranges therebetween).

In various embodiments, the extruding step is carried out at a temperature between about 25 degrees Celsius to about 42 degrees Celsius (e.g., about 25, 26, 27, 28, 29, 30, 31, 32, 33, 34, 35, 36, 37, 38, 39, 40, 41, or 42 degrees Celsius inclusive of all ranges therebetween).

In various embodiments, the fibrin microthreads are formed by using a dispensing apparatus, the dispensing apparatus having a first reservoir containing fibrinogen, a second reservoir containing a molecule capable of forming fibrin from the fibrinogen (e.g. thrombin), a blending connector fluidically coupled to the first reservoir and the second reservoir, and a lumen containing device fluidically coupled to the blending connector. A first volume of fibrinogen from the first reservoir is transferred to the blending connector; a second volume of the molecule capable of forming fibrin from the fibrinogen is transferred from the second reservoir to the blending connector; the first volume of fibrinogen and the second volume of the molecule forming a mixture in the blending connector. The mixture from the blending connector is transferred to the lumen containing device in an aqueous bath and the distal end of the lumen containing device is moved through the aqueous bath at a first velocity and the mixture is extruded from the distal end of the lumen containing device into the aqueous bath at a second velocity while moving the distal end of the lumen containing device through the aqueous bath. In some embodiments, the ratio of the second velocity to the first velocity is in the range of about 1.5 to about 6 (e.g. about 1.5, or about 2.0, or about 2.5, or about 3.0, or about 3.5, or about 4.0, or about 4.5, or about 5.0, or about 5.5, or about 6.0). In some embodiments, the mixture is incubated in the aqueous bath for a predetermined incubation time to form the fibrin microthread (e.g. about 1 to about 30 min., or about 5 to about 25 min., or about 10 to about 20 min., or about 15 to about 20 min., or about 1 min., or about 5 min., or about 10 min., or about 15 min., or about 20 min., or about 25 min., or about 30 min).

In certain embodiments, the fibrin microthreads are formed by combining a first volume of fibrinogen with a second volume of a molecule capable of forming fibrin from fibrinogen (e.g. thrombin) to form a mixture. The mixture is transferred to a lumen containing device and a distal end of the lumen containing device is disposed in an aqueous bath. The mixture is extruded from the distal end of the lumen containing device while moving the distal end through the aqueous bath. The fibrin microthreads substantially form in the aqueous bath and are then removed from the aqueous bath. As described herein, a plurality of threads formed in this manner are optionally formed into yarns in the bath or outside of the bath after drying.

In an illustrative embodiment, the fibrin microthreads described herein can be formed using a mechanical process. For example, in some embodiments, the fibrin microthreads can be formed by combining fibrinogen and a molecule capable of forming fibrin form the fibrinogen, for example, the enzyme thrombin, to form a mixture. The mixture can be transferred to a lumen containing device, for example, a tube or a conduit. A distal end of the lumen containing device can be disposed in an aqueous bath. The mixture can be extruded from the distal end of the lumen containing device, while moving the distal end of the lumen containing device through the aqueous bath. This deposits the mixture in the aqueous bath that is allowed to form into the fibrin microthread, for example, after incubating for a predetermined time.

Fibrin is a proteolytic cleavage product of fibrinogen. Fibrinogen, a soluble protein typically present in human blood plasma at concentrations between about 2.5 and 3.0 g/L, is intimately involved in a number of physiological processes including homeostasis, angiogenesis, inflammation, and wound healing. Fibrinogen is 340,000 Da hexameric glycoprotein composed of pairs of three different subunit polypeptides, Aα, Bβ, and γ, linked together by a total of 29 disulfide bonds. During the normal course of blood coagulation, the enzyme thrombin cleaves small peptides from the Aα and Bβ chains of fibrinogen to generate the insoluble fibrin monomer. The fibrin monomers self-assemble in a staggered overlapping fashion through non-covalent, electrostatic interactions to form protofibrils; the protofibrils further assemble laterally into thicker fibers that ultimately intertwine to produce a clot.

Fibrinogen is expressed primarily in the liver, although low levels of extrahepatic synthesis have been reported for other tissues, including bone marrow, brain; lung and intestines. The thrombin catalyzed conversion of fibrinogen to fibrin is common to all extant vertebrates and accordingly, the amino acid sequence of fibrinogen is highly conserved evolutionarily. Each polypeptide subunit is the product of a separate but closely linked gene; multiple isoforms and sequence variants have been identified for the subunits. Amino acid sequences for the fibrinogen subunits are in the public domain. The fibrinogen Aα polypeptide is also known as fibrinogen a chain polypeptide; fibrinogen a chain precursor; Fib2; MGC119422; MGC119423; and MGC119425. The fibrinogen Bβ polypeptide is also known as fibrinogen β chain polypeptide; fibrinogen β chain preproprotein; MGC104327; and MGC120405 and the fibrinogen y polypeptide is also known as fibrinogen γ chain polypeptide and fibrinogen γ chain precursor.

Any form of fibrinogen that retains the ability to function (e.g., retains sufficient activity to form fibrin in the presence of a molecule capable of forming fibrin from fibrinogen) may be used in the manufacture of the fibrin microthreads. The fibrinogen can be a human fibrinogen or a fibrinogen of a non-human primate, a domesticated animal, a bovine tissue, or a rodent. The fibrinogen can be obtained from a naturally occurring source or recombinantly produced. The amino acid sequence of fibrinogen subunit polypeptides can be identical to a standard reference sequence in the public domain. In some embodiments, the fibrinogen can include biologically active variants of a fibrinogen subunit polypeptide. For example, a biologically active variant of a fibrinogen subunit polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a fibrinogen subunit polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. Alternatively, any of the components can contain mutations such as deletions, additions, or substitutions.

The fibrinogen may be partially or substantially pure. The term “substantially pure” with respect to fibrinogen refers to fibrinogen that has been separated from cellular components by which it is naturally accompanied, such that it is at least 60% (e.g., 70%, 80%, 90%, 95%, or 99%), by weight, free from polypeptides and naturally-occurring organic molecules with which it is naturally associated. In general, a substantially pure polypeptide will yield a single major band on a non-reducing polyacrylamide gel. A substantially pure polypeptide provided herein can be obtained by, for example, extraction from a natural source (e.g., blood or blood plasma from human or animal sources), non-human primates (e.g., monkeys, baboons, or chimpanzees), pigs, cows, horses, goats, sheep, dogs, cats, rabbits, guinea pigs, gerbils, hamsters, rats, or mice), chemical synthesis, or by recombinant production in a host cell.

The fibrinogen can include post-translational modifications, i.e., chemical modification of the polypeptide after its synthesis. Chemical modifications can be naturally occurring modifications made in vivo following translation of the mRNA encoding the fibrinogen polypeptide subunits or synthetic modifications made in vitro. A polypeptide can include one or more post-translational modifications, in any combination of naturally occurring, i.e., in vivo, and synthetic modifications made in vitro. Examples of post-translational modifications include glycosylation (e.g., addition of a glycosyl group to either asparagine, hydroxy lysine, serine or threonine residues to generate a glycoprotein or glycopeptides). Glycosylation is typically classified based on the amino acid through which the saccharide linkage occurs and can include: N-linked glycosylation to the amide nitrogen of asparagines side chains, O-linked glycosylation to the hydroxyl oxygen of serine and threonine side chains, and C-mannosylation. Other examples of post-translation modification include, but are not limited to, acetylation, for example, the addition of an acetyl group, typically at the N-terminus of a polypeptide; alkylation, for example, the addition of an alkyl group; isoprenylation, for example, the addition of an isoprenoid group; lipoylation, for example, attachment of a lipoate moeity; phosphorylation, for example, addition of a phosphate group to serine, tyrosine, threonine or histidine; and biotinylation, for example, acylation of lysine or other reactive amino acid residues with a biotin molecule.

Fibrinogen can be purified using any standard method including, but not limited to, methods based on fibrinogen's low solubility in various solvents, its isoelectric point, fractionation, centrifugation, and chromatograph. Such methods can include, for example, gel filtration, ion exchange chromatography, reverse-phase HPLC, and immunoaffinity purification. partially or substantially purified fibrinogen can also be obtained from commercial sources, including for example Sigma, St. Louis, Mo., Hematologic Technologies, Inc. Essex Junction, Vt., or Aniara Corp. Mason, Ohio.

Any concentration of fibrinogen that results in fibrin microthread formation can be used. For example, in some embodiments, the concentration of fibrinogen can be about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, or about 120 mg/ml. In some embodiments, the concentration of fibrinogen can be about 1% Clottable/mL, or about 1.5% Clottable/mL, or about 2% Clottable/mL, or about 2.5% Clottable/mL, or about 3% Clottable/mL, or about 3.5% Clottable/mL, or about 4% Clottable/mL, or about 4.5% Clottable/mL, or about 5% Clottable/mL, or about 7.5% Clottable/mL, or about 10% Clottable/m L.

Fibrinogen can also be produced by recombinant DNA techniques. Nucleic acid segments encoding the fibrinogen polypeptide subunits can be operably linked in a vector that includes the requisite regulatory elements, for example, promoter sequences, transcription initiation sequences, and enhancer sequences, for expression in prokaryotic or eukaryotic cells. Methods well known to those skilled in the art can be used to construct expression vectors containing relevant coding sequences and appropriate transcriptional/translational control signals. Alternatively, suitable vector systems can be purchased from commercial sources. The nucleic acid molecules can be synthesized (e.g., by phosphoramidite based synthesis) or obtained from a biological cell, such as the cell of a mammal. The nucleic acids can be those of mammal, for example, humans, a non-human primates, cattle, horses, pigs, sheep, goats, dogs, cats, rabbits, guinea pigs, hamsters, rats, or mice.

The molecule capable of forming fibrin from fibrinogen can be any naturally occurring or synthetic molecule capable of cleaving fibrinogen, thereby producing fibrin. For example, in some embodiments, the molecule can include thrombin. The aqueous bath can include any aqueous medium that is compatible with the activity of the fibrin-forming enzyme for example, thrombin. Suitable aqueous mediums can include buffer systems such as, for example, HEPES-buffered saline, tris-buffered saline, phosphate buffered saline, MES, PIPES. The buffer may also include a divalent cation such as, for example, CaCl₂. The pH of the aqueous bath can vary from less than about 8.5 (e.g., less than about 8.3, 8.1, 8.0, 7.9, 7.8, 7.7, 7.6, 7.5, 7.4, 7.3, 7.2, 7.1, 7.0, 6.9, 6.8, 6.7, inclusive of all ranges therebetween) to more than about 5.5 (e.g., more than about 5.7, 5.8, 6.0, 6.1, 6.2, 6.3, 6.4, 6.5, 6.6, 6.7, 6.8, inclusive of all ranges therebetween). In various embodiments, the temperature of the aqueous bath can be any temperature compatible with fibrin polymerization and can vary from, for example, less than about 40 degrees Celsius (e.g., less than about 39, 38, 37, 36, 35, 34, 33, 32, 31, 30, 29, 28 degrees Celsius, inclusive of all ranges therebetween) to more than about 18 degrees Celsius (e.g., more than about 19, 20, 21, 22, 23, 24, 25, 26, 27 degrees Celsius, inclusive of all ranges therebetween).

Any of the concentrations of fibrinogen, the molecule capable of forming fibrin from fibrinogen, the pH of the aqueous medium, and the swelling temperature may be adjusted to achieve optimal fibrin microthread formation. For example, fibrinogen from different sources, for example, different mammalian species or different isoforms of fibrinogen from the same species, may require different cleavage conditions in order to synthesize fibrin microthreads of requisite tensile strength or tissue regeneration properties.

In some embodiments, the mixture of fibrinogen and molecule capable of forming fibrin from fibrinogen can be incubated in the aqueous bath for a predetermined incubation time to allow fibrin to substantially form in the aqueous bath. For example, the mixture can be incubated for at least about 1 minute, 2 minutes, 3 minutes, 5 minutes, 10 minutes, 15 minutes, 20 minutes, 30 minutes, 45 minutes, 1 hour, 2 hours, 3 hours, about 4 hours, about 5 hours, or even more.

In some embodiments, the incubation step includes features that prevent the extruded solution from adhering or substantially adhering to the surface of the vessel in which the aqueous bath is contained. Any method that is compatible with fibrin polymerization may be used. For example, the vessel can include one or more materials having an extremely low coefficient of friction to provide a non-stick surface, e.g., polytetrafluoroethylene (Teflon®), fluorinated ethylene-propylene (FEP) and perfluoroalkoxy polymer resin (PFA). Alternatively or in addition, the aqueous bath can include one or more surfactants, detergents or emulsifying agents, for example, Pluronic® surfactants (BASF) polyethylene glycol, or tri-ethylene glycol. The appropriate concentration of such reagent will vary according to the nature of the reagent. Alternatively or in addition, the aqueous bath is physically agitated.

The fibrin microthreads can be recovered from the aqueous bath and dried. The fibrin microthreads can be dried in air, or any other gas, for example, nitrogen. The drying temperature may be ambient temperature, for example, about 25 degrees Celsius, or a temperature that is mildly elevated relative to ambient temperature, for example, in the range of about 28 degrees Celsius to about 44 degrees Celsius (e.g., about 29, 30, 31, 32, 33, 34, 35, 36, 37, 38, 39, 40, 41, 42, or about 43 degrees Celsius inclusive of all ranges therebetween).

In some embodiments, methods for producing multifilament fibrin microthreads are provided. The method comprises an extrusion step in which the microthreads are extruded into a bath (e.g., aqueous buffer bath), followed by drying the individual microthreads. Varying numbers of dried threads can then be twisted together to produce a multifilament microthread yarn.

In an alternative embodiment, methods for producing monofilament fibrin microthreads are provided. The method comprises an extrusion step in which the microthreads are extruded into a bath as described herein. Prior to removal from the extrusion bath, varying numbers of microthreads (e.g. more than 2, more than 5, more than 6, more than 7, more than 8, more than 9, or more than 10 such as 3, 4, 5, 6, 7, 8, 9, 10, 11, 12, 13, 14, or 15) are pulled together in the bath (e.g. buffer solution) to form a single, cohesive thread. The thread is then removed from the bath and allowed to dry.

In certain embodiments, multiple layers of fibrin are deposited into the bath to form multifilament or monofilament microthreads. For example, about 1 layer, 2 layers, 3 layers, 4 layers, 5 layers, 6 layers, 7 layers, 8 layers, 9 layers, 10 layers, 11 layers, 12 layers, 13 layers, 14 layers, 15 layers, 16 layers, 17 layers, 18 layers, 19 layers, or 20 layers may be deposited into the bath to form multifilament or monofilament fibrin microthreads. In an embodiment, monofilament fibrin microtheads are formed.

In various embodiments, the time between each layer deposition is from about 1 second to about 10 minutes. For example, the time between each layer deposition is from about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 35 seconds, about 40 seconds, about 45 seconds, about 50 seconds, about 55 seconds, about 60 seconds, about 70 seconds, about 80 seconds, about 90 seconds, about 100 seconds, about 110 seconds, about 120 seconds, about 150 seconds, about 3 minutes, about 4 minutes, about 5 minutes, about 6 minutes, about 7 minutes, about 8 minutes, about 9 minutes, or about 10 minutes.

In various embodiments, thrombin is used for forming fibrin microthreads. The thrombin concentration may be in the range of about 1 U/mL to about 100 U/mL, about 1 U/mL to about 95 U/mL, about 1 U/mL to about 90 U/mL, about 1 U/mL to about 85 U/mL, about 1 U/mL to about 80 U/mL, about 1 U/mL to about 75 U/mL, about 1 U/mL to about 70 U/mL, about 1 U/mL to about 65 U/mL, about 1 U/mL to about 60 U/mL, about 1 U/mL to about 55 U/mL, about 1 U/mL to about 60 U/mL, about 1 U/mL to about 55 U/mL, about 1 U/mL to about 50 U/mL, about 1 U/mL to about 45 U/mL, about 1 U/mL to about 40 U/mL, about 1 U/mL to about 35 U/mL, about 1 U/mL to about 30 U/mL, about 1 U/mL to about 25 U/mL, about 1 U/mL to about 20 U/mL, about 1 U/mL to about 15 U/mL, about 1 U/mL to about 10 U/mL, about 1 U/mL to about 9 U/mL, about 1 U/mL to about 8 U/mL, about 1 U/mL to about 7 U/mL, about 1 U/mL to about 6 U/mL, about 1 U/mL to about 5 U/mL, about 1 U/mL to about 4 U/mL, about 1 U/mL to about 3 U/mL, or about 1 U/mL to about 2 U/mL. For example, the thrombin concentration may be about 1 U/mL, about 2 U/mL, about 3 U/mL, about 4 U/mL, about 5 U/mL, about 6 U/mL, about 7 U/mL, about 8 U/mL, about 9 U/mL, about 10 U/mL, about 11 U/mL, about 12 U/mL, about 13 U/mL, about 14 U/mL, about 15 U/mL, about 16 U/mL, about 17 U/mL, about 18 U/mL, about 19 U/mL, about 20 U/mL. about 25 U/mL, about 30 U/mL, about 35 U/mL, about 40 U/mL, about 45 U/mL, about 50 U/mL, about 55 U/mL, or about 60 U/mL, inclusive of all values and ranges therebetween.

In various embodiments, CaCl₂ is included in the bath used for extrusion. The CaCl₂ concentration may be in the range of about 0.1 mM to about 20 mM, about 0.1 mM to about 15 mM, about 0.1 mM to about 10 mM, about 0.1 mM to about 9 mM, about 0.1 mM to about 8 mM, about 0.1 mM to about 7 mM, about 0.1 mM to about 6 mM, about 0.1 mM to about 5 mM, about 0.1 mM to about 4 mM, about 0.1 mM to about 3 mM, about 0.1 mM to about 2 mM, or about 0.1 mM to about 1 mM. For example, the CaCl2 concentration may be about 0.1 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM, inclusive of all values and ranges therebetween.

In various embodiments, an additional step of rehydrating and stretching the fibrin microthreads is included. In such embodiments, the fibrin microthreads are extruded into a bath where they are maintained at 100% of their initial length and allowed to dry. Subsequently, the fibrin microthreads are rehydrated in, for example, distilled water, and stretched to at least about 110%, about 120%, about 130%, about 140%, about 150%, about 160%, about 170%, about 180%, about 190%, about 200%, about 250%, about 300%, about 400%, or about 500% of their initial length. The stretched fibrin microthreads are then allowed to dry. It is believed that such an additional drying, rehydrating, and stretching step may enhance the mechanical strength of the fibrin microthread.

Alternatively, the fibrin microthreads are stretched directly after extrusion. For example, the fibrin microthreads are stretched in bath directly after extrusion. In such embodiments, the fibrin microthreads are stretched within about 60 minutes of initial extrusion. For example, the fibrin microthreads are stretched within about 60 minutes, about 55 minutes, about 50 minutes, about 45 minutes, about 40 minutes, about 35 minutes, about 30 minutes, about 25 minutes, about 20 minutes, about 15 minutes, about 10 minutes, about 9 minutes, about 8 minutes, about 7 minutes, about 6 minutes, about 5 minutes, about 4 minutes, about 3 minutes, about 2 minutes, or about 1 minute of initial extrusion.

In certain embodiments, the fibrin microthreads (for example, multifilament or monofilament fibrin microthreads) are twisted following stretching. In an embodiment, the stretching improves cross-sectional uniformity. In various embodiments, twisting may be performed at a level of between 0.1 twists/cm to around 3 twists/cm. For example, twisting may be performed at 0.1 twists/cm, 0.5 twists/cm, 1 twists/cm, 1.5 twists/cm, 2 twists/cm, 2.5 twists/cm, or 3 twists/cm.

To form sutures using either the multifilament or monofilament fibrin microthreads, dried threads are inserted into the bore hold of a standard drilled-end surgical needle and crimped into place. In various embodiments, the sutures are readily attached to needles of various types as described herein (for example, bored-end needle or eyelet-style needle). Optionally, the sutures are packaged and sterilized, for example, via a 12-hour ethylene oxide cycle. In some embodiments, the needle is a straight, ¼ circle, ⅜ circle, ½ circle (e.g. CT, CT-1, CT-2 and CT-3), ⅝ circle, compound curved, half curved (ski), half curved at both ends (canoe), taper, cutting, reverse cutting, trocar point, blunt point, or a side cutting needle. By way of illustration ETHICON needles (NOVARTIS), LOOK needles (SURGICAL SPECIALTIES), or SYNETURE needles (COVIDIEN) may be used in the present invention.

In certain embodiments, the fibrin microthreads (for example, multifilament or monofilament fibrin microthreads) are twisted following needle attachment. In various embodiments, twisting may be performed at a level of between 0.1 twists/cm to around 3 twists/cm. For example, twisting may be performed at 0.1 twists/cm, 0.5 twists/cm, 1 twists/cm, 1.5 twists/cm, 2 twists/cm, 2.5 twists/cm, or 3 twists/cm.

In some embodiments, a fibrin microthread can be chemically cross-linked (e.g. covalently linked) to itself and/or other fibrin microthreads. One suitable method of cross-linking is exposure to ultra-violet (UV) light. Levels of UV exposure may vary according to the size and configuration of the fibrin microthreads and can range for example, from a calculated total energy of about 4 to about 100 J/cm², e.g., about 4.5, 5.0, 8.0, 10.0, 15.0 17.1, 20.0 25.0, 30.0 40.0 50.0 60.0. 70.0 80.0, 90.0, 100.0 J/cm². Cross-linking can also be carried out using chemical cross-linking agents. Chemical cross-linking agents can be homo-bifunctional (the same chemical reaction takes place at each end of the linker) or hetero-bifunctional (different chemical reactions take place at the ends of the linker). The chemistries available for such linking reactions include, but are not limited to, reactivity with sulfhydryl, amino, carboxyl, diol, aldehyde, ketone, or other reactive groups using electrophilic or nucleophilic chemistries, as well as photochemical cross-linkers using alkyl or aromatic azido or carbonyl radicals. Examples of chemical cross-linking agents include, without limitation, glutaraldehyde, carbodiimides, bisdiazobenzidine, and N-maleimidobenzoyl-N-hydroxysuccinimide ester. Chemical cross-linkers are widely available from commercial sources (e.g., Pierce Biotechnology (Rockford, Ill.); Invitrogen (Carlsbad, Calif.); Sigma-Aldrich (St. Louis, Mo.); and US Biological (Swampscott, Mass.). Particularly suitable cross-linking reagents include 1-ethyl-3-[3-dimethylaminopropyl]carbodiimide hydrochloride (EDAC), and N-hydroxysulfosuccinimide (NHS). The duration of the cross-linking reaction may vary according to the cross-linking agent that is used, the reaction temperature and the tensile strength desired.

In some embodiments, the fibrin microthreads can be submitted to one or more treatments to diminish the bioburden. These treatments can be configured to inactivate or kill substantially all microorganisms (e.g., bacteria, fungi (including yeasts), and/or viruses) in the fibrin microthreads. Said another way, the treatments can be used to sterilize the fibrin microthreads. Suitable sterilization treatments can include ultra-violet light, autoclave, ethylene oxide, gamma radiation, electron beam radiation, supercritical carbon dioxide sterilization, any other sterilization treatment process or combination thereof.

The fibrin microthreads described herein can be hydrated before performing wound ligation. Hydration can be performed in any suitable aqueous medium, for example, a buffer solution such as, for example, phosphate buffered saline, HEPES-buffered saline, tris-buffered saline, MES, PIPES, Lactated Ringer's solution, Dulbecco's minimum essential media (DMEM), Ham's F-10 media, Ham's F-12 media, minimum essential media (MEM), any other suitable aqueous solution or combination thereof. The hydration can swell the fibrin microthreads. In some embodiments, the hydrated fibrin microthreads can have a tensile strength can be about 1 MPa, about 2 MPa, about 3 MPa, about 4 MPa, about 5 MPa, about 6 MPa, about 7 MPa, about 8 MPa, about 9 MPa, about 10 MPa, about 15 MPa, about 20 MPa, or even higher, inclusive of all ranges therebetween.

In some embodiments, a therapeutic agent can be incorporated in the fibrin microthreads. The therapeutic agent can include, for example, a growth factor, a protein, a chemotherapeutic agent, a vitamin, a mineral, an antimicrobial agent, a small organic molecule, or a biological cell. The therapeutic agent can be incorporated in the fibrin microthread using any suitable process such as, for example, covalent bonding to the fibrin microthread, surface adsorption, or physical incorporation during the preparation of the fibrinogen or fibrin-forming solutions, during mixing of the fibrinogen and the molecule capable of forming fibrin, during post-formation adsorption while still in a forming buffer, and/or during absorption during a subsequent hydration process.

In illustrative embodiments, a therapeutic agent can be incorporated in the fibrin microthread by (a) extruding the therapeutic agent with the fibrinogen and a molecule capable of forming fibrin from fibrinogen thereby producing a fibrin microthread comprising the therapeutic agent or (b) associating the therapeutic agent with a formed fibrin microthread. In illustrative embodiments, associating the therapeutic agent with a formed fibrin microthread can be achieved by covalently bonding the therapeutic agent to the fibrin microthread (by, for example, exposing the therapeutic agent and the fibrin microthread to a ligase that, for example, generates a carbon-oxygen bond, a carbon-sulfur bond, a carbon-nitrogen bond, or a carbon-carbon bond between the therapeutic agent and the fibrin microthread). One can also expose the therapeutic agent and the fibrin microthread to a crosslinking agent (e.g., a chemical crosslinking agent or ultraviolet radiation).

In some embodiments, the therapeutic agents can include agents that promote tissue regeneration. In such embodiments, the therapeutic agents can include growth factors including, for example, cytokines and interleukins, extracellular matrix proteins and/or biologically active fragments thereof (e.g., RGD-containing peptides), blood and serum proteins, nucleic acids, hormones, vitamins, chemotherapeutics, antibiotics and cells. These agents can be incorporated into the fibrin microthreads prior to implantation in a host tissue. Alternatively, the therapeutic agents can be injected into or applied to fibrin microthreads already implanted in the host tissue. These agents can be administered singly or in combination. For example, the fibrin microthreads can be used to deliver cells, growth factors and small molecule therapeutics concurrently, or to deliver cells plus growth factors, or cells plus small molecule therapeutics, or growth factors plus small molecule therapeutics.

Growth factors that can be incorporated into the fibrin microthreads can include any of a wide range of cell growth factors, angiogenic factors, differentiation factors, cytokines, hormones, and chemokines known in the art. Growth factors can be polypeptides that include the entire amino acid sequence of a growth factor, a peptide that corresponds to only a segment of the amino acid sequence of the native growth factor, or a peptide that is derived from the native sequence that retains the bioactive properties of the native growth factor. The growth factor can be a cytokine or interleukin. Any combination of two or more of the growth factors can be included in the fibrin microthreads. Examples of relevant factors include vascular endothelial cell growth factors (VEGF) (e.g., VEGF A, B, C, D, and E), platelet-derived growth factor (PDGF), insulinlike growth factor (IGF) I and IGF-II, interferons (IFN) (e.g., IFN -α, β or γ), fibroblast growth factors (FGF) (e.g., FGF 1, FGF-2, FGF-3, FGF-4-FGF-10), epidermal growth factor, keratinocyte growth factor, transforming growth factors (TGF) (e.g., TGFα or β), tumor necrosis factor-a, an interleukin (IL) (e.g., IL-I, IL-2, 11-17 -IL-18), Osterix, Hedgehogs (e.g., sonic or desert), SOX9, bone morphogenetic proteins (BMP's), in particular, BMP 2, 4, 6, and (BMP-7 is also called OP-1), parathyroid hormone, calcitonin prostaglandins, or ascorbic acid.

In some embodiments, the therapeutic agents can include proteins. In some embodiments, the proteins can be delivered to the host tissue by including in the fibrin microthreads any one of the following: (a) expression vectors (e.g., plasmids or viral vectors) containing nucleic acid sequences encoding any one or more of the above factors that are proteins; or (b) cells that have been transfected or transduced (stably or transiently) with such expression vectors. Such transfected or transduced cells will preferably be derived from, or histocompatible with, the host tissue. However, it is possible that only short exposure to the factor is required and thus histo-incompatible cells can also be used. Other useful proteins can include, without limitation, hormones, extracellular antibodies, extracellular matrix proteins, and/or biologically active fragments thereof (e.g., RGD-containing peptides) or other blood and serum proteins, e.g., fibronectin, albumin, thrombospondin, Von Willebrand factor and fibulin.

In some embodiments, the therapeutic agents can include small molecule drugs, thus facilitating localized drug delivery. Long-term systemic administration of antibiotics may only be partially effective against such subclinical infections. Incorporation of antimicrobial agents into the fibrin microthreads can provide local high concentrations of antibiotics, thus minimizing the risk of adverse effects associated with long term high systemic doses. Examples of antibiotics include, without limitation, any representative classes of antibiotics, e.g., 1) aminoglycosides, such as gentamycin, kanamycin, neomycin, streptomycin or tobramycin; 2) cephalosporins, such as cefaclor, cefadroxil or cefotaxime; 3) macrolides, such as azithromycin, clarithromycin, or erythromycin; 4) penicillins, such as amoxicillin, carbenicillin or penicillin; 5) peptides, such as bacitracin, polymixin B or vancomycin; 6) quinolones, such as ciprofloxacin, levofloxacin, or enoxacin; 7) sulfonamides, such as sulfamethazole, sulfacetimide; or sulfamethoxazole; 8) tetracyclines, such as doxycycline, minocycline or tetracycline; 8) other antibiotics with diverse mechanisms of action such as rifampin, chloramphenicol, or nitrofuratoin. Other antimicrobial agents, e.g., antifungal agents and antiviral agents can also be used.

In some embodiments, the therapeutic agents can include anti-inflammatory agents and/or anti-proliferative agents. In some embodiments, the anti-inflammatory agents can include, non-steroidal anti-inflammatory drugs (NSAIDs) such as, for example, aspirin, choline and magnesium salicylates, celecoxib, diclofenac potassium, diclofenac sodium, diclofenac sodium with misoprostol, diflunisal, etodolac, fenoprofen, flurbiprofen, ibuprofen, indomethacin, ketoprofen, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, valdecoxib, any other suitable NSAID or a combination thereof. In some embodiments, the anti-inflammatory agents can include corticosteroids such as, for example, hydrocortisone, hydrocortisone acetate, cortisone acetate, tixocortol pivalate, prednisolone, methylprednisolone, methylprednisolone aceponate, prednisone, triamcinolone acetonide, triamcinolone alcohol, mometasone, mometasone furoate, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-valerate, halometasone, alclometasone dipropionate, betamethasone valerate, betamethasone dipropionate, prednicarbate, clobetasone-17-butyrate, clobetasol-17-propionate, fluocortolone caproate, fluocortolone pivalate, fluprednidene acetate, hydrocortisone-17-butyrate, hydrocortisone-17-aceponate, hydrocortisone-17-buteprate, hydrocortisone valerate, flurandrenolide, triamcinolone acetonide, ciclesonide, halobetasol, diflorasone diacetate, fluocinonide, halicinonide, amcinonide, desoximetasone, fluticasone propionate, betamethasone dipropionate, desonide, alclometasone dipropionate, clobetasol propionate, prednicarbate, any other suitable corticosteroid or a combination thereof.

In some embodiments, the therapeutic agents can include cells, for examples stem cells. For example, histocompatible, viable cells can be included in the fibrin microthreads to produce a permanently accepted graft that may be remodeled by the host tissue. Cells can be derived from the intended recipient or an allogeneic donor. Cell types with which the fibrin microthreads can be repopulated include, but are not limited to, embryonic stem cells (ESC), adult or embryonic mesenchymal stem cells (MSC), monocytes, hematopoetic stem cells, gingival epithelial cells, endothelial cells, fibroblasts, or periodontal ligament stem cells, prochondroblasts, chondroblasts, chondrocytes, pro-osteoblasts, osteocytes, or osteoclast. Any combination of two or more of these cell types (e.g., two, three, four, five, six, seven, eight, nine, or ten) may be used to repopulate the biocompatible tissue repair composition. Methods for isolating specific cell types are well known in the art. Donor cells may be used directly after harvest or they can be cultured in vitro using standard tissue culture techniques. Donor cells can be infused or included in the fibrin microthreads in situ just prior to placing of the biocompatible tissue repair composition in a mammalian subject. Donor cells can also be co-cultured with the fibrin microthreads using standard tissue culture methods known to those in the art.

Embodiments of the fibrin microthreads described herein can be configured in any suitable form, shape, or size corresponding to the size and shape of the tissue repair that is desired. The fibrin microthreads may be organized by basic bundling, braiding, twisting, or cabling depending upon specific needs. For example, in some embodiments a plurality of the fibrin microthreads can be braided or woven together to form a rope or a yarn. In some embodiments, the plurality of the fibrin microthreads can be coupled together to form woven or non-woven meshes, dressing, bandage, gauze, web, film, patch, sheath or graft for application to or implantation in a tissue, for example, for tissue ligation. In various embodiments, the fibrin microthreads can be associated with a substrate (by, for example, coating, wrapping, or otherwise permanently or non-permanently associating the microthreads with the substrate). In certain embodiments, the substrate can be a woven or non-woven mesh, a surgical needle, a surgical pin, a surgical screw, a surgical plate, a patch, a dressing, a bandage, or a natural or mechanical valve.

In various embodiments, the fibrin microthread sutures of the invention comprise filaments of polymerized fibrin that are generally cylindrical in shape. In an embodiment, the fibrin microthread sutures comprise multiple filaments (i.e., multi-filament). The diameter of the filaments is generally in the range of from about 1 μM to about 300 μM. For example, the diameter of the filaments is from about 1 μM to about 290 μM, from about 1 μM to about 280 μM, from about 1 μM to about 270 μM, from about 1 μM to about 260 μM, from about 1 μM to about 250 μM, from about 1 μM to about 240 μM, from about 1 μM to about 230 μM, from about 1 μM to about 220 μM, from about 1 μM to about 210 μM, from about 1 μM to about 200 μM, 1 μM to about 190 μM, from about 1 μM to about 180 μM, from about 1 μM to about 170 μM, from about 1 μM to about 160 μM, from about 1 μM to about 150 μM, from about 1 μM to about 140 μM, from about 1 μM to about 130 μM, from about 1 μM to about 120 μM, from about 1 μM to about 110 μM, from about 1 μM to about 100 μM from about 1 μM to about 90 μM, from about 1 μM to about 80 μM, from about 1 μM to about 70 μM, from about 1 μM to about 60 μM, from about 1 μM to about 50 μM, from about 1 μM to about 40 μM, from about 1 μM to about 30 μM, from about 1 μM to about 20 μM, from about 1 μM to about 10 μM, or from about 1 μM to about 5 μM, inclusive of all values and ranges therebetween. In various embodiments, the diameter of the filaments is about 200 μM, about 190 μM, about 180 μM, about 170 μM, about 160 μM, about 150 μM, about 140 μM, from about 130 μM, about 120 μM, about 110 μM, about 100 μM, about 90 μM, about 80 μM, about 70 μM, about 60 μM, about 50 μM, about 40 μM, about 30 μM, about 20 μM, about 10 μM, or about 5 μM, inclusive of all values and ranges therebetween.

In another embodiment, the fibrin microthread sutures comprise a single filament (i.e., monofilament). Without wishing to be bound by theory, it is believed that monofilament fibrin microthread sutures may reduce the risks for infections. The diameter of the monofilament is generally in the range of from about 100 μM to about 2,000 μM. For example, the diameter of the filaments is from about 100 μM to about 1,900 μM, from about 100 μM to about 1,800 μM, from about 100 μM to about 1,700 μM, from about 100 μM to about 1,600 μM, from about 100 μM to about 1,500 μM, from about 100 μM to about 1,400 μM, from about 100 μM to about 1,300 μM, from about 100 μM to about 1,200 μM, from about 100 μM to about 1,100 μM, from about 100 μM to about 1,000 μM, from about 100 μM to about 900 μM, from about 100 μM to about 800 μM, from about 100 μM to about 700 μM, from about 100 μM to about 600 μM, from about 100 μM to about 500 μM, from about 100 μM to about 400 μM, from about 100 μM to about 300 μM, from about 100 μM to about 200 μM, or from about 100 μM to about 150 μM, inclusive of all values and ranges therebetween. In various embodiments, the diameter of the monofilament is about 2,000 μM, about 1,900 μM, about 1,800 μM, about 1,700 μM, about 1,600 μM, about 1,500 μM, about 1,400 μM, from about 1,300 μM, about 1,200 μM, about 1,100 μM, about 1,000 μM, about 900 μM, about 800 μM, about 700 μM, about 600 μM, about 500 μM, about 400 μM, about 300 μM, about 200 μM, or about 100 μM, inclusive of all values and ranges therebetween.

In some embodiments, the yarns or monofilaments may be further organized into a mesh or “sheet” configuration. These mesh devices can leverage the adjustable stiffness and elasticity of the fibrin yarns or monofilaments and further augment these characteristics through knit or weaving patterns as appropriate. In certain embodiments, the meshes or “sheets” may be useful in support of soft tissue repairs requiring a fast absorbing, mechanically compliant reinforcement or overlay material. In certain embodiments, the device may be useful for retention of delivered materials in a specified tissue or organ by formation of a pouch or patch. In some embodiments, the device may be useful for mechanical reinforcement over larger tissue defects as an overlay which may be sutured or otherwise affixed in place.

In some embodiments, textile engineering techniques are used to generate a three-dimensional weave or mesh. In certain embodiments, the three-dimensional weave or mesh may be used as an organized tissue void filling substrate. Sutured into place at its borders, the three-dimensional weave or mesh can provide initial mechanical support to a tissue defect, prevent abscess formation, and provide organizational cues to ingrowing host cells.

In some embodiments, the yarns can be organized into a reinforcing or isolating sleeve. The sleeve can provide mechanically compliant reinforcement around a healing tissue. The mechanical compliance of the sleeve, particularly used in conjunction with a mechanically compliant suture along with the biocompatibility of fibrin, provides for a minimally inflammatory environment with minimal likelihood for a device-induced adverse outcome such as scar formation, occlusion, or wound breakdown.

In some embodiments, additional filaments of other material types may be integrated into the fibrin yarns or threads as parallel filaments, a core material, or a sheathing. These materials may include, but are not limited to, a range of conventional synthetic or biosynthetic fibers (e.g., polypropylene, nylon, PLGA) and natural fibers (e.g., collagen, silk, elastin). These materials may be added to modulate Young's modulus, ultimate tensile strength (UTS), absorption rate, and strength retention profiles. These materials may also be added as yarns integrated into a composite woven or knit device to alter the aforementioned properties in addition to burst mechanics and directional device tensile mechanics. A synthetic fiber can include, but is not limited to, an aliphatic polyester, a poly(amino acid), poly(propylene fumarate), a copoly(ether-ester), a polyalkylene oxalate, a polyamide, a tyrosine-derived polycarbonate, a poly(iminocarbonate), a polyorthoester, a polyoxaester, a polyamidoester, a polyoxaester containing one or more amine groups, a poly(anhydride), a polyphosphazine, a polyurethane, a biosynthetic polymer, or a combination thereof. The aliphatic polyester can include, but is not limited to, homopolymers or copolymers of lactides; glycolides; ε-caprolactone; hydroxybuterate; hydroxyvalerate; 1,4-dioxepan-2-one; 1,5,8,12-tetraoxyacyclotetradecane-7,14-dione; 1,5-dioxepan-2-one; 6,6-dimethyl-1,4-dioxan-2-one; 2,5-diketomorpholine; p-dioxanone(1,4-dioxan-2-one); trimethylene carbonate(1,3-dioxan-2-one); alkyl derivatives of trimethylene carbonate; δ-valerolactone; β-butyrolactone; γ-butyrolactone, ε-decalactone, pivalolactone, α,α-diethylpropiolactone, ethylene carbonate, ethylene oxalate; 3-methyl-1,4-dioxane-2,5-dione; 3,3-diethyl-1,4-dioxan-2,5-dione; 6,8-dioxabicycloctane-7-one; or combinations thereof. A biosynthetic fiber can include, but is not limited to, a polymer comprising a sequence found in collagen, elastin, thrombin, fibronectin, a starch, gelatin, alginate, pectin, oxidized cellulose, chitin, chitosan, tropoelastin, hyaluronic acid, a ribonucleic acid, a deoxyridonucleic acid, a polypeptide, a polysaccharide, or a combination thereof. A natural fiber can include, but is not limited to, collagen or a collagen-based material, hyaluronic acid or a hyaluronic acid-based material, cellulose or a cellulose-based material, silk and combinations thereof.

In various embodiments, the step of combining the fibrin microthread with a microthread comprising a non-fibrin polymer can include, for example, weaving the fibrin microthread and the microthread comprising the non-fibrin polymer, bundling the fibrin microthread and the microthread comprising the non-fibrin polymer to form a filament, or tying or interlacing the fibrin microthread and the microthread comprising the non-fibrin polymer to form a non-woven mesh. The fibrin microthread and the microthread comprising the non-fibrin polymer can be coextruded. For example, a fibrin microthread can be extruded through one orifice into a receptacle and a non-fibrin microthread can be extruded through a second orifice into the same or a different receptacle.

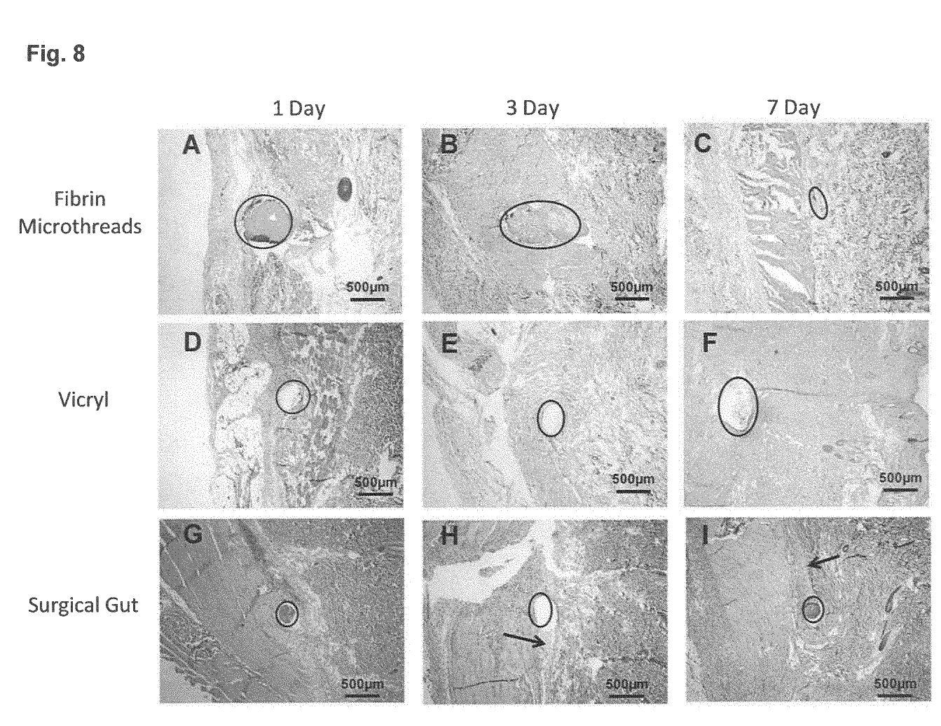

The present fibrin microthreads find use in a variety of suture techniques, including but not limited to, the simple interrupted stitch, the vertical and horizontal mattress stitch, the running or continuous stitch, the chest drain stitch, the corner stitch, the purse-string suture, the FIG. 8 stitch, and the subcuticular stitch.

Further, the present fibrin microthreads may be used in combination with topical cyanoacrylate adhesives (“liquid stitches”), a.k.a. medicinal grade super glue in wound closure. Cyanoacrylate is the generic name for cyanoacrylate based fast-acting glues such as methyl-2-cyanoacrylate, ethyl-2-cyanoacrylate (commonly sold under trade names like SUPERGLUE and KRAZY GLUE) and n-butyl-cyanoacrylate. Skin glues like INDERMIL and HISTOACRYL, composed of n-butyl cyanoacrylate, are also useful. Further, 2-octyl cyanoacrylate (e.g. LIQUIBAND, SURGISEAL, FLORASEAL, and DERMABOND) may be used.

Also, in some embodiments, the present fibrin microthreads may be used in combination with various skin closure tapes to ensure proper wound closure with the characteristics described herein. For instance, PROXI STRIP or a polyester fiber strip (e.g. MERSILENE) may be used.

In some embodiments, embodiments of the fibrin microthreads described herein can be included in kits that can simplify the delivery of the fibrin microthread to the host tissue. An illustrative kit of the invention described herein can include a predetermined length of the fibrin microthread. The fibrin microthread can be wound, coiled, or disposed on a spool. A needle can be coupled to a distal end of the fibrin microthread. The needle can include a straight, ¼ circle, ⅜ circle, ½ circle, ⅝ circle, compound curved, half curved (ski), half curved at both ends (canoe), taper, cutting, reverse cutting, trocar point, blunt point, side cutting needle, or any other suitable needle commonly known in the arts. The fibrin microthread can be included in the kit in a dehydrated state. A container, for example, a pouch, a vial, a prefilled syringe, a bottle, a carton, or any other suitable container that includes a volume of a hydrating solution can be included in the kit for hydrating the fibrin microthread before a ligating procedure. Suitable hydrating solutions can include, for example, a buffer solution (e.g., phosphate buffered saline, HEPES-buffered saline, tris-buffered saline, MES, PIPES), Lactated Ringer's solution, Dulbecco's minimum essential media (DMEM), Ham's F-10 media, Ham's F-12 media, minimum essential media (MEM), any other suitable aqueous solution or combination thereof.

The kit can also include apparatus and devices for performing incisions in a host tissue or assist in implantation of the fibrin microthreads into the host tissue. For example, the kit can include one or more scalpels, for example, a number 6 scalpel, a number 9 scalpel, a number 10 scalpel, a number 15 scalpel, any other suitable scalpel or a combination thereof. The scalpel can be provided with the scalpel handle and the scalpel blade coupled together, or disposed separately in the kit such that a user can couple the scalpel blade to the scalpel handle before the surgical procedure. The kit can include a needle holder (e.g., a Mayo-Hegar needle holder, castroviejo needle holder, etc.) and one or more forceps (e.g., artery forceps, atraumatic forceps, biopsy forceps, bulldog forceps, dermal forceps, microsurgery forceps, tissue forceps, any other forceps or combination thereof) to allow facile manipulation of the needle and/or the fibrin microthread for implantation into the host tissue. The kit can also include a surgical probe and a skin holder. Furthermore, scissors can be included in the kit, for example, to cut a length of the fibrin microthreads.

The kit can include medicaments or compositions to disinfect at least a portion of the host tissue. For example, the kit can include alcohol (e.g., ethyl alcohol), alcohol pads, alcohol wipes, alcohol swabs, anti-septic towels, iodine wipes, benzoin wipes, etc. The kit can include compositions or materials for dressing the ligated site such as, for example, anti-biotic medicaments (e.g., anti-biotic creams, ointments, or lotions), anti-inflammatory medicaments (e.g., anti-inflammatory creams, ointments, or lotions), surgical pads, cotton gauze, wound closure strips, bandages, surgical tape, or any other medicament or articles that can be used for dressing a ligated site. The kit can further include a label or printed instructions instructing the user on the use of any component included in the kit. The components of the kit can be disposed in a suitable housing for example, a bi-fold bag, a tri-fold bag, or any suitable housing for housing the components of the kit. Each of the components of the kit can be sterilized before disposing in the kit. Suitable sterilization procedures can include, for example, autoclaving, ultra-violet treatment, ethylene oxide treatment, any other sterilization treatment or combination thereof.

The following show various examples of fibrin microthread sutures used for suturing incision wounds such that there is reduced inflammation, little or no scarring, and fast absorption of the fibrin microthreads in the host tissue. These examples are only for illustrative purposes and are not intended to limit the scope of the present disclosure.

### EXAMPLES

### Example 1

### Dry and Hydrated Fibrin Microthread

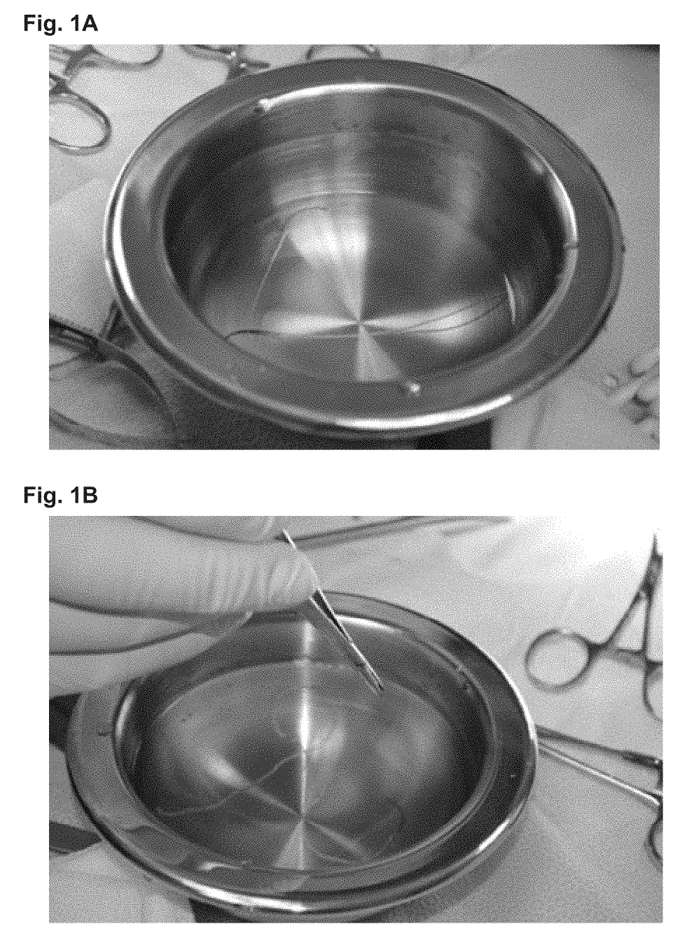

FIG. 1A shows a dry fibrin microthread disposed in a container. The fibrin microthread was formed by combining fibrinogen and thrombin, which were disposed in internal volumes of two separate luer locked syringes, in a blending connector to form a mixture of the fibrinogen (48 mg/mL clottable fibrinogen protein dissolved in a solution of 20 mM pH 7.4 HEPES buffered saline) and the thrombin (6 U/mL dissolved in a solution of 3 mM pH 7.4 HEPES with 34 mM calcium chloride). The mixture was communicated to a tube coupled to the blending connector. A distal end of the tube was disposed in an aqueous bath, an excess of pH 7.2, 10 mM HEPES solution. The aqueous bath was maintained at ambient temperature, nominally 20 degrees Celsius and a pH of about 7.2. The distal end of the tube was moved in the aqueous bath using a robotic manipulator and simultaneously the mixture was extruded through the distal end of the tube into the aqueous bath. The mixture disposed in the aqueous bath was incubated for a time of about 30 minutes such that the fibrin microthread formed substantially in the aqueous bath. The fibrin microthread was removed form the aqueous bath and dried in air under ambient conditions. The fibrin microthread was then hydrated in lactated Ringer's solution which included about 130 mEq/L of sodium ions, 109 mEq/L of chloride ions, 28 mEq/L of lactate, 4 mEq/L of potassium ions, and 2.7 mEq/L of calcium ions, for about 10 minutes before being used as ligating suture for incision wounds as described herein. FIG. 1B shows the fibrin microthread of FIG. 1A after hydration for 10 minutes in the lactated Ringer's solution.

### Example 2

### Fibrin Microthread Sutures for Ligating Dorsal Skin Incisions

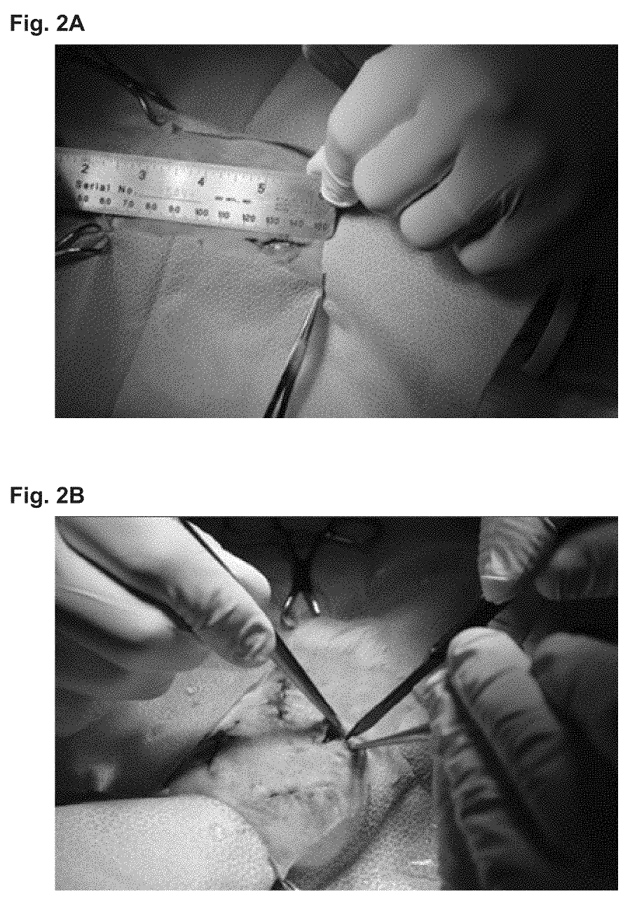

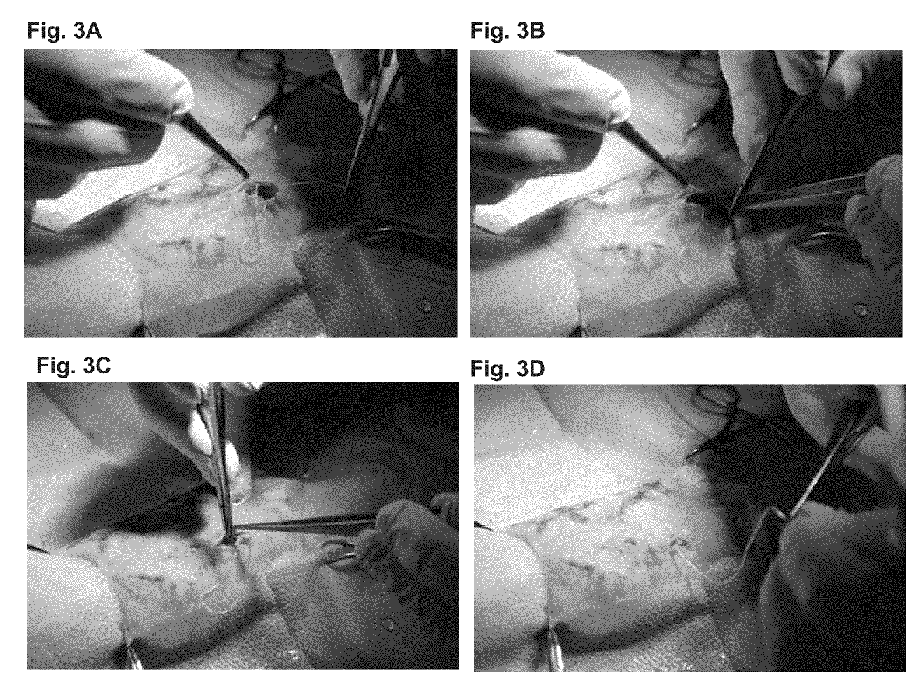

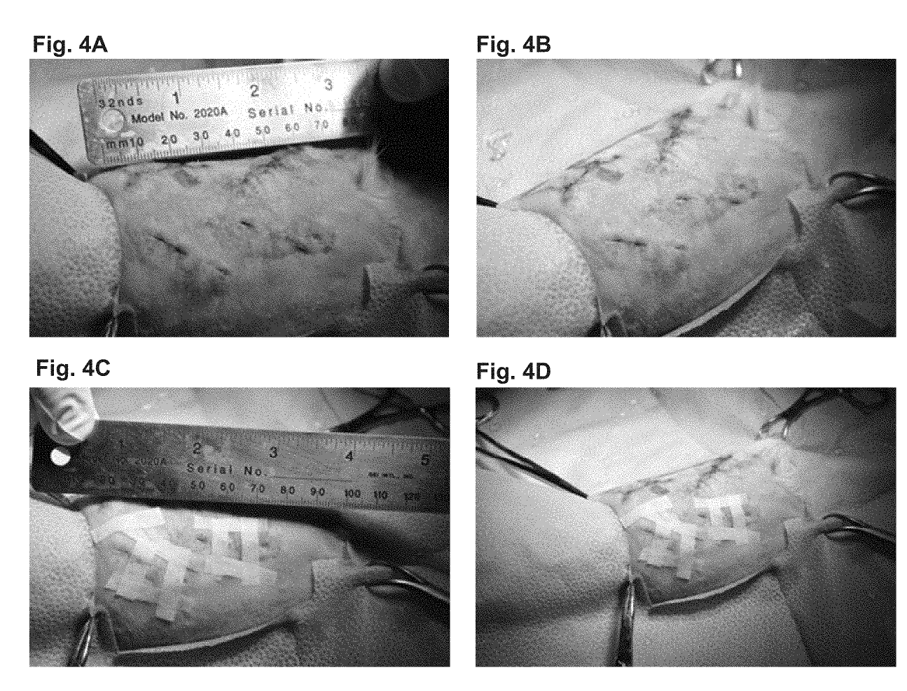

Sprague Dawley rats were anesthetized by dosing intraperitoneally with an anesthetic cocktail including 50 mg/kg of ketamine and 4 mg/kg of xylazine. As necessary, inhaled isoflurane was used to adjust anesthesia during the procedure. A plurality of three centimeter incision wounds were made in dorsal skin of the rats using a surgical scalpel. FIG. 2A shows a 3 cm dorsal skin incision made in a rat and FIG. 2B shows a plurality of dorsal skin incisions made in the rat. The incisions were ligated with a simple running intradermal suture pattern using either fibrin microthreads, a VICRYL® suture, or a surgical gut suture. FIGS. 3A-C show a dorsal skin incision in various stages of ligation by the fibrin microthread. FIGS. 4A and 4B show the incision wounds after ligation by the fibrin microthread. FIGS. 4C and 4D show the ligated incision wounds dressed with a surgical dressing. Initial pilot animals demonstrated an inclination to chew at their wound sites irrespective of material used for repair and as a result, E-collars were placed on the rats to prevent the rats from chewing on the sutures, thereby preventing non-device-related wound dehiscence.

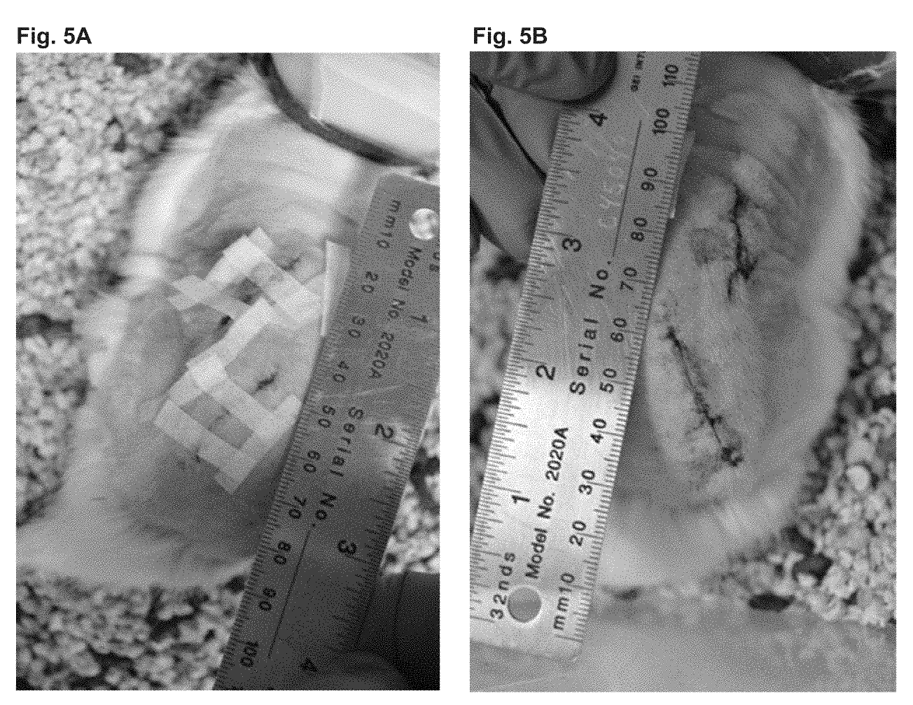

FIG. 5A shows the incision wound ligated by the fibrin microthread, and FIG. 5B shows an incision wound ligated with the VICRYL® suture, 1 day after surgery. Both the fibrin microthread and the VICRYL® suture ligated incision wounds remain substantially closed one day after surgery. The fibrin microthread ligated incision visually appeared to have less inflammation then the VICRYL® suture ligated incision.

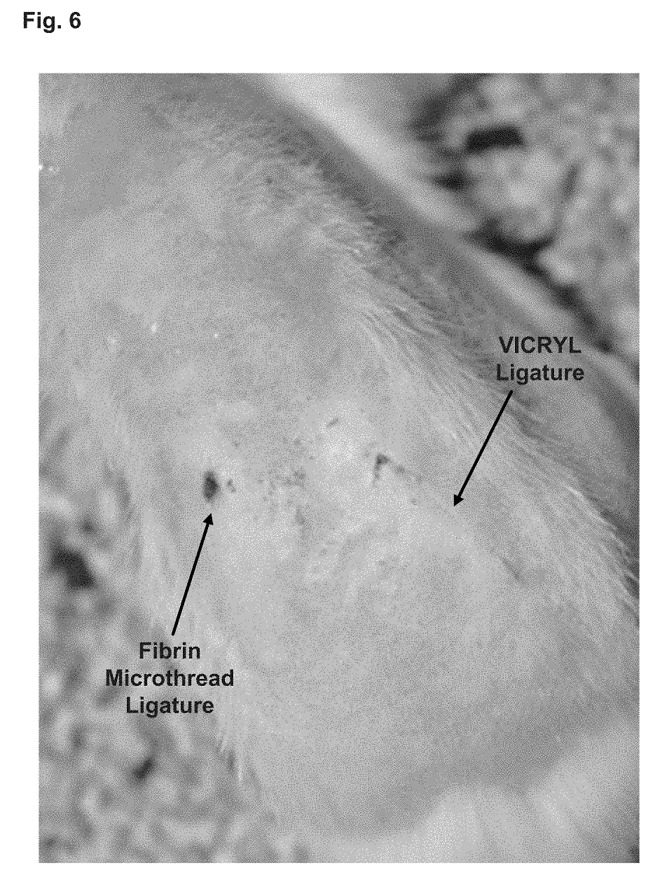

FIG. 6 shows a fibrin microthread ligated incision wound and a VICRYL® suture ligated incision wound on the dorsal skin of the rat one week after surgery. Slight evidence of the incision wound was observable for both the incision sites.

### Example 3

### Histopatholoqical Staining and Scoring of Suture Site

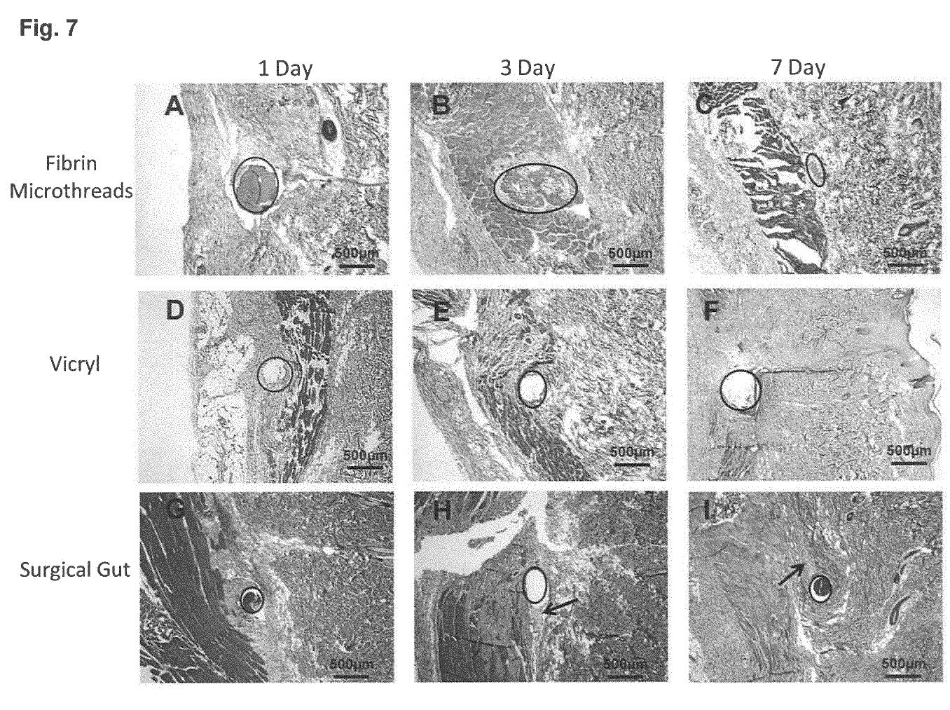

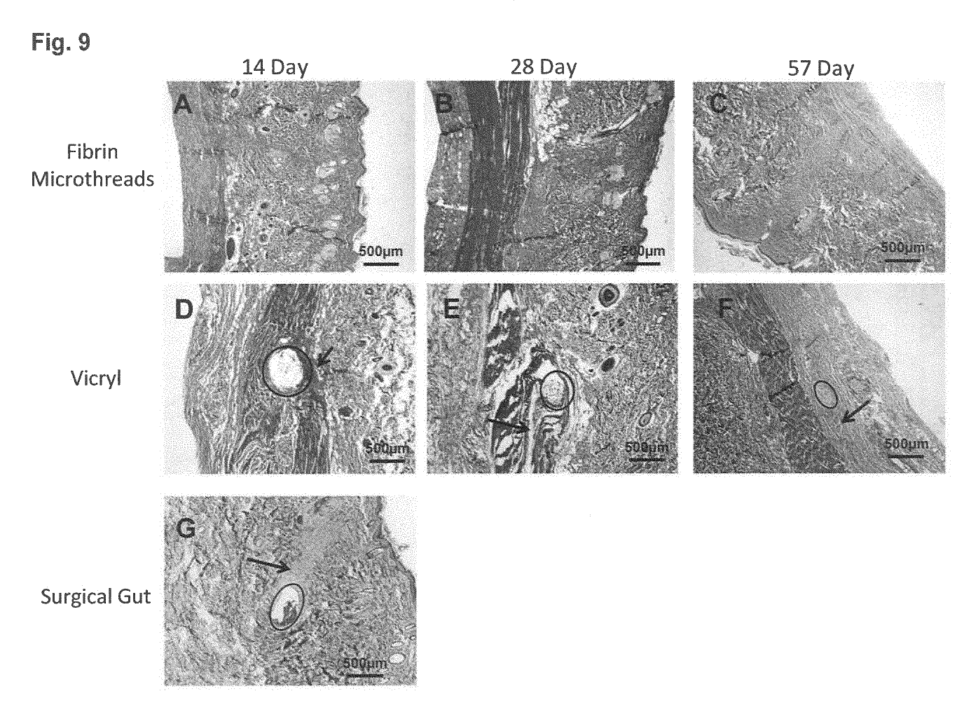

To observe and quantify the severity of inflammation, scarring (collagen deposition), and absorption of the fibrin microthread in the rat tissue, histopathological staining was used. Acute (short term) as well as long term (chronic histology) was studied. The dermis of the rats corresponding to the incision sites was harvested at 1 day, 3 days, and 7 days post surgery to study acute histology, and harvested at 14 days, 28 days and 57 days post surgery to study chronic histology. The harvested tissue was stained with trichrome blue, and hematoxylin and eosin (H&E) stain. FIG. 7 (Trichrome stained) and 8 (H&E stained) show acute histology, and FIG. 9 (Trichrome stained) and 10 (H&E stained) show early chronic histology of the dermis of the rats explanted from the portion of the skins which included the incision wound ligated with fibrin microthreads, VICRYL® or surgical gut sutures. The implanted sutures are indicated by black circles, and extensive collagen deposition consistent with implant encapsulation and fibrosis is indicated by black arrows.

FIG. 7 panel A, B and C show images of H&E-stained histology of the dermis proximal to incision wounds explanted from a first set of rats, that were ligated with fibrin microthreads, at day 1, day 3 and day 7 post surgery, respectively. FIG. 8 panel A, B and C show images of Trichrome-stained histology of the dermis proximal to incision wounds explanted from a second set of rats, that were ligated with fibrin microthreads, also explanted at day 1, day 3 and day 7 post surgery, respectively. The fibrin microthreads did not generate significant hypercellular response on day 1, typically seen as large quantities of closely clustered dark purple dots near and around the implant or injured tissue in H&E stains or by light purple regions in Trichrome. By day 3 the fibrin microthreads were moderately infiltrated by immune cells with some light neocollagen deposition occurring peripherally. The cell infiltration is evidenced by a series of purple streaks running into the interior of the circled implant in both the H&E and Trichrome histology samples. The neocollagen deposition is evidenced by the light blue stain occurring circumferentially around the circled implant in the Trichrome image. The fibrin microthread became substantially acellular and non-fibrotic again by day 7 evidenced by the lack of visible cell nuclei and neocollagen deposition either within or peripheral to the circled implant material. Furthermore, the fibrin microthreads had substantially decreased in volume by day 7 as compared to day 1 clearly indicating that the fibrin microthread had started to absorb in the host tissue.

FIG. 7 panel D, E and F show images of H&E-stained histology of the dermis proximal to incision wounds explanted from a first set of rats that were ligated with VICRYL® sutures, at day 1, day 3 and day 7 post surgery, respectively. Similarly, FIG. 8 panel D, E and F show images of Trichrome-stained histology of the dermis proximal to incision wounds explanted from a second set of rats that were also ligated with VICRYL® sutures, and also explanted at day 1, day 3 and day 7 post surgery, respectively. The VICRYL® sutures demonstrated an insignificant host response at day 1, exhibiting minimal nuclear staining proximal to the implant. By day 3 and day 7, increasing migration of host immune and inflammatory cells to the area are seen occurring as a ring of cells and new collagen around the implanted device in E (purple under H&E, dark blue under Trichrome) and significant areas of purple stained cells and collagen around the implant (pale purple in H&E, dark purple in Trichrome) in F. While some of the hypercellularity in F is clearly associated with the incision wound (visible as a purple track progressing from right to left mid-way down the image), there is substantial nuclear staining circumferentially around the circled implant material. This is indicative of an inflammatory response which may progress to encapsulation over time. Furthermore, increased collagen staining around the implant perimeter indicates developing fibrosis. Moreover, there was no noticeable decrease in the implant size from day 1 to day 7, indicating that very little or no absorption of the VICRYL® suture had occurred in the host tissue.

FIG. 7 panel G, H and I show images of H&E-stained histology of the dermis proximal to incision wounds explanted from a first set of rats, that were ligated with surgical gut sutures, at day 1, day 3, and day 7 post surgery, respectively. Similarly, FIG. 8 panel G, H and I show images of Trichrome-stained histology of the dermis proximal to incision wounds explanted from a second set of rats, that were also ligated with surgical gut sutures, and also explanted at day 1, day 3 and day 7 post surgery, respectively. The surgical gut sutures also demonstrated an insignificant host response at day 1, but by day 3 substantial evidence of the implant encapsulation was observable as evident from a purple track running obliquely to the plane of the image, shown by black arrows in FIG. 7 panel H and I, and FIG. 8 panel H and I (pale purple in H&E, dark purple in Trichrome). The encapsulation behavior was suggested by the apparent hypercellularity and neo-collagen deposition around the implant. The characteristic purple color of these capsules is caused by the large number of nuclei and prominent collagen staining in the track. Furthermore, there was no noticeable decrease in the implant size from day 1 to day 7, indicating that very little or no absorption of the surgical gut suture had occurred in the host tissue.

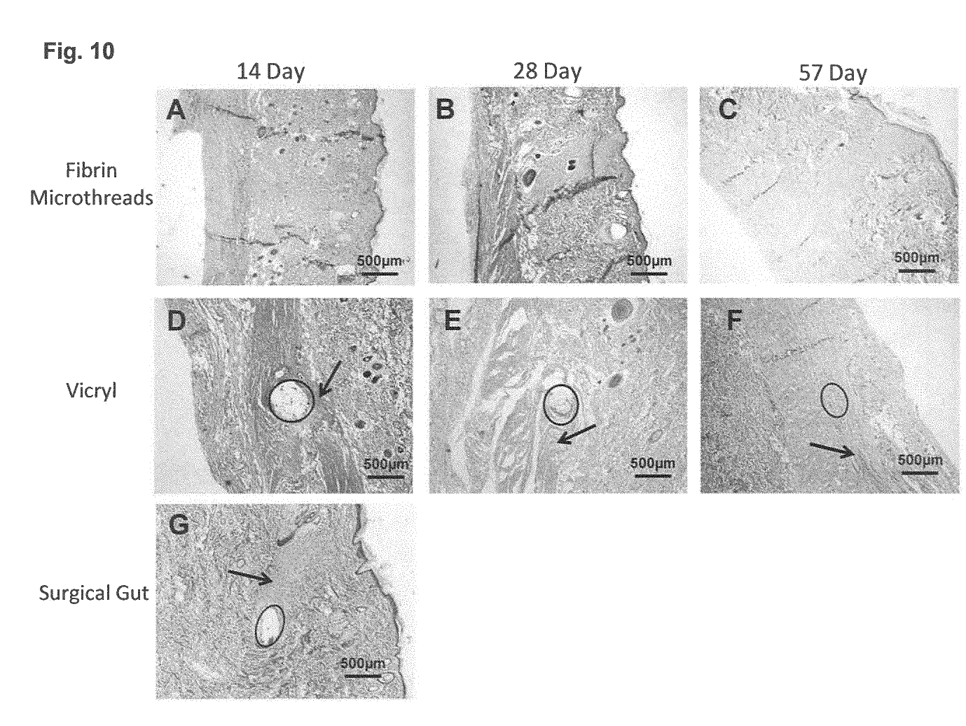

FIG. 9 panel A, B and C show images of H&E-stained histology of the dermis proximal to incision wounds explanted from the first set of rats, that were ligated with fibrin microthreads, at 14 days, 28 days, and 57 days post surgery, respectively. Similarly, FIG. 10 panel A, B and C show images of Trichrome-stained histology of the dermis proximal to incision wounds explanted from a second set of rats, that were ligated with fibrin microthreads, and also explanted at 14 days, 28 days, and 57 days post surgery, respectively. No residual evidence of the fibrin microthread could be observed on day 14, clearly showing that the fibrin microthread had substantially absorbed in the host tissue within 14 days of implantation. Furthermore, no disruption of the tissue beyond the healed incision, evidenced by disruptions in the regular pattern of the reticular dermis appearing as a blue or light blue smudge under Trichrome or a dark pink or light purple smudge under H&E was observed.

FIG. 9 panel D, E and F show images of H&E-stained histology of the dermis proximal to incision wounds explanted from the first set of rats that were ligated with VICRYL® sutures, at day 14, day 27, and day 57 post surgery, respectively. Similarly, FIG. 10 panel D, E and F show images of Trichrome-stained histology of the dermis proximal to incision wounds explanted from a second set of rats, that were also ligated with VICRYL® sutures, and also explanted at day 14, day 27, and day 51 post surgery, respectively. The VICRYL® sutures histology illustrated a substantially more encapsulation behavior in the host tissue which was best evidenced by the strong blue staining in the Trichrome at the peripheral portions of the suture site at day 14 and day 28. By day 57 host cells had infiltrated the VICRYL® suture and were continuing to deposit collagen within the suture implant boundaries evidenced by purple staining within the suture borders seen in both H&E and Trichrome images. Furthermore, there was only minimal decrease in the size of the implanted VICRYL® suture after 57 days.

FIG. 9 panel G shows an image of H&E-stained histology of the dermis proximal to an incision wound explanted from a first of rat, and FIG. 10 panel G shows an image of Trichrome-stained histology of the dermis proximal to incision wounds explanted from a second rat, that were ligated with surgical gut sutures and explanted at day 14 post surgery. The surgical gut suture continued to present a fibrotic encapsulation response from the host with a track running obliquely to the plane of the image, as shown by the black arrow in panel G and best indicated by a light blue streak in the Trichrome staining. The encapsulation behavior was associated with hypercellularity and neo-collagen deposition around the suture site evidenced by the pale purple staining circumferential to the implant in the H&E staining. No noticeable decrease in the implant size was observed from day 1 to day 14.

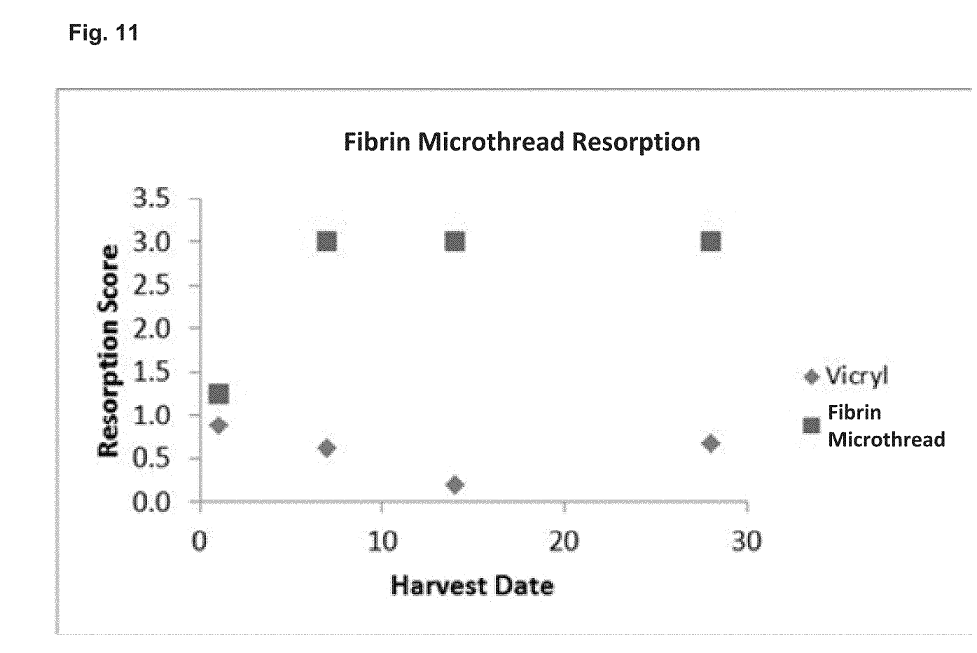

Following explant and processing, histology slides were scored by a certified histopathologist on the HAI scale to provide semi-quantitative data regarding the response of the host to each of the sutures. A minimum of four sites were evaluated for each suture. FIG. 11 shows a plot of the resorption score of the fibrin microthreads and the VICRYL® sutures after day 1, day 7, day 14, and day 28 post-surgery, obtained form the HAI scoring of the host tissue slides. After day 7 post-surgery, the fibrin microthreads demonstrated a substantially higher resorption score relative to the VICRYL® sutures which indicates that the fibrin microthreads absorb much faster in the host tissue relative to the VICRYL® sutures.

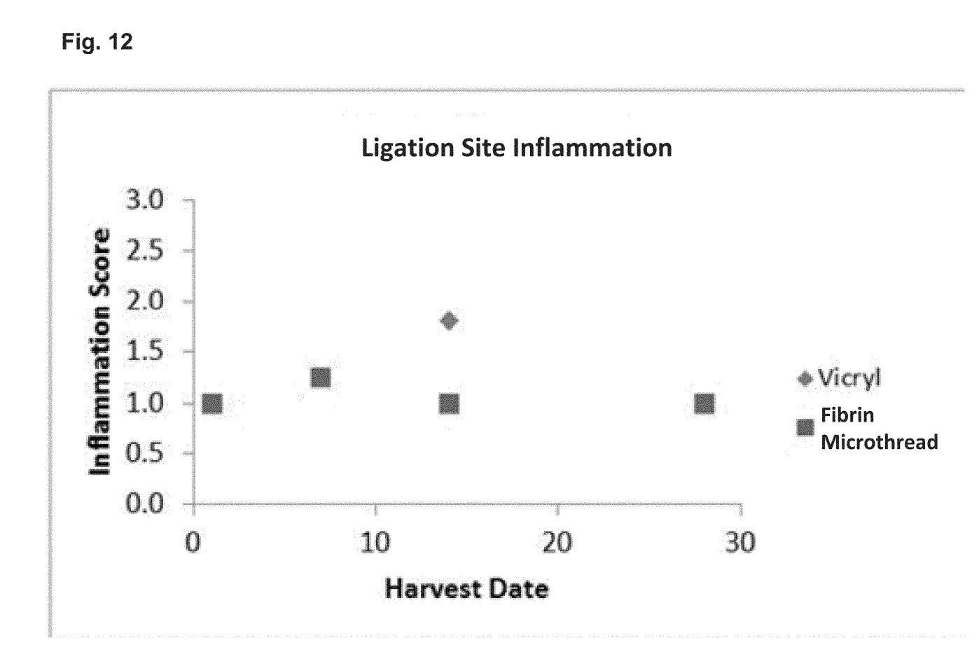

FIG. 12 shows a plot of the ligation site inflammation scores caused by the fibrin microthreads and the VICRYL® sutures after day 1, day 7, day 14, and day 28 post-surgery, obtained form the HAI scoring of the host tissue slides. The fibrin microthreads and the VICRYL® sutures had low inflammation scores indicating that the inflammation at the ligation site elicited by each of the fibrin microthreads and the VICRYL® sutures was minimal.

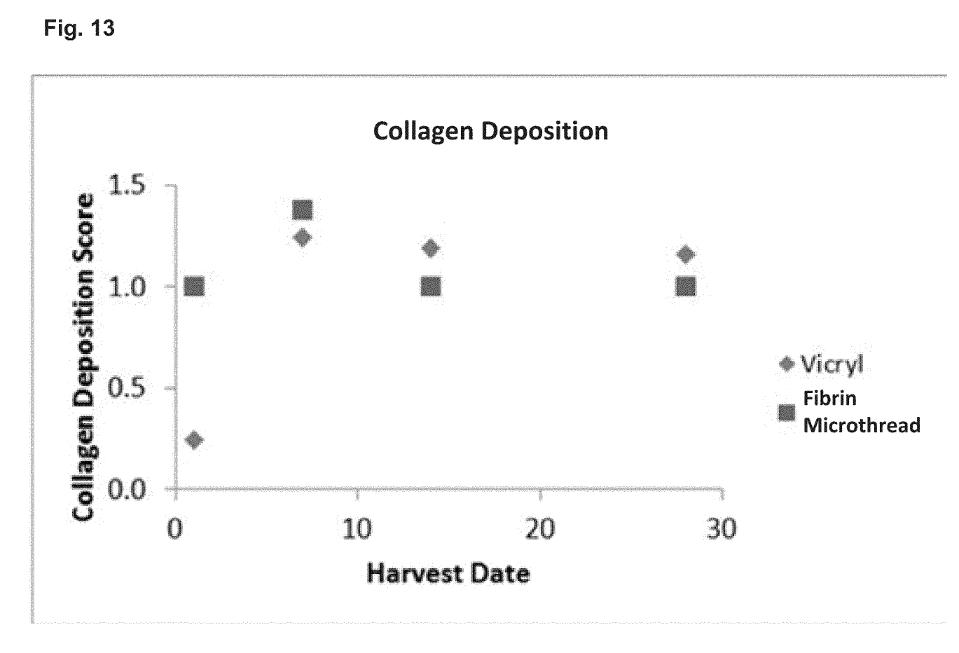

FIG. 13 shows a plot of the collagen deposition score because of the fibrin microthreads and the VICRYL® sutures after day 1, day 7, day 14, and day 28 post-surgery, obtained form the HAI scoring of the host tissue slides. Neither of the fibrin microthreads and the VICRYL® elicited substantial collagen deposition response, with the fibrin microthreads scoring slightly better than the VICRYL® sutures.

The histology scores of the fibrin microthreads and VICRYL® and are summarized in Table 1.

TABLE 1
Histological Scores of Host Response to Sutures

| | | | | | | Collagen | Overall | | Number |
|---|---|---|---|---|---|---|---|---|---|
| | Neutro- | Eosino- | Macro- | Lympho- | Fibro- | Depo- | Inflam- | Resorp- | of |
| Suture Type | phils | phils | phages | cytes | cytes | sition | mation | tion | Samples |
| VICRYL ® day 1 | 0.9 | 0.6 | 0.3 | 0.3 | 0.3 | 0.3 | 1.0 | 0.9 | 8 |
| VICRYL ® day 7 | 0.6 | 0.1 | 1.3 | 1.1 | 1.1 | 1.3 | 1.3 | 0.6 | 8 |
| VICRYL ® day 14 | 0.2 | 0 | 1.0 | 1.0 | 0.2 | 1.2 | 1.8 | 0.2 | 5 |
| VICRYL ® day 28 | 0.2 | 0 | 1.0 | 1.0 | 0.2 | 1.2 | 1.0 | 0.7 | 6 |
| Fibrin microthread Day 1 | 1.3 | 0 | 1.0 | 1.0 | 1.0 | 1.0 | 1.0 | 1.3 | 8 |
| Fibrin microthread Day 7 | 0.5 | 0 | 1.0 | 1.0 | 1.1 | 1.4 | 1.3 | 3.0 | 8 |
| Fibrin microthread Day 14 | 0 | 0 | 1.0 | 1.0 | 0 | 1.0 | 1.0 | 3.0 | 5 |
| Fibrin microthread Day 28 | 0.2 | 0 | 1.0 | 0.8 | 0 | 1.0 | 1.0 | 3.0 | 6 |

Score: 0 = None; 1 = Trace; 2 = Apparent; 3 = Prominent

The histopathological scores were substantially consistent with the histology observations. The fibrin microthreads elicited an overall inflammation response which was more muted than the inflammation response elicited by the VICRYL® suture. Minimal amount of neutrophils and eosinophils were observed on day 1, while a slightly higher number of macrophages, lymphocytes, and plasma cells were observed at day 1. Collagen deposition remained at trace levels over a period of 28 days post surgery. Furthermore, a substantial portion of the fibrin microthreads were absorbed in the host tissue within 7 days post surgery and substantially all of the fibrin microthreads were absorbed in the host tissue by day 14 post surgery. The VICRYL® suture also elicited a minimal immune response from the neutrophils and the eosinophils. However, the VICRYL® sutures resulted in a prolonged fibrotic response encouraging fibrotic collagen deposition, inflammation, and fibrocyte presence even 28 days post surgery. Furthermore, the VICRYL® sutures were still visible in the host tissue after day 28, while only a small portion of the VICRYL® was absorbed in the host tissue.

### Example 4

### Fibrin Suture Closure Strength in Sprague Dawley Rat Dorsal Skin

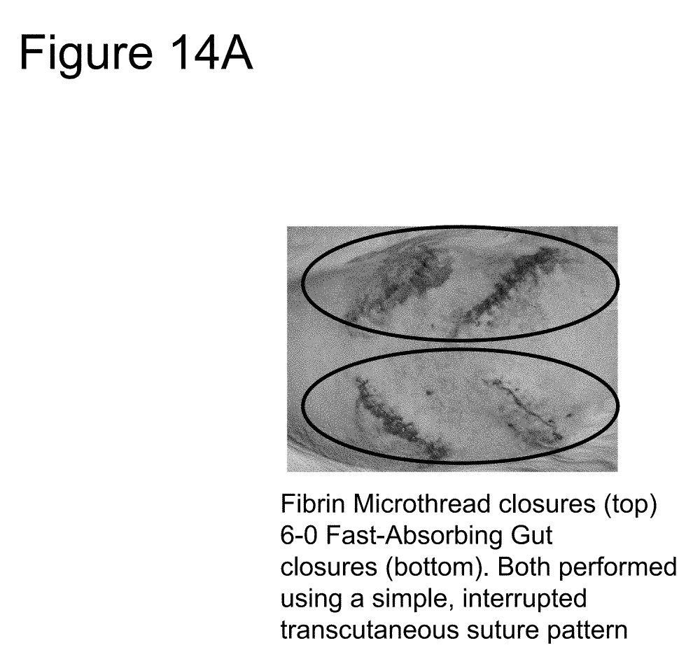

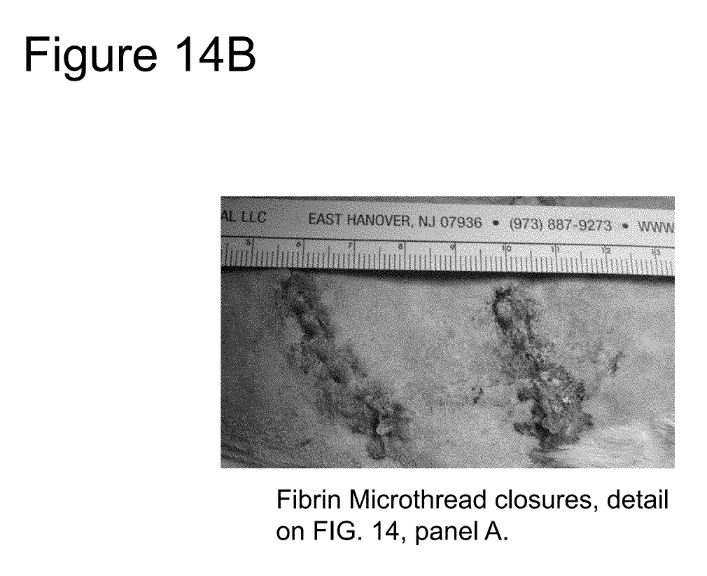

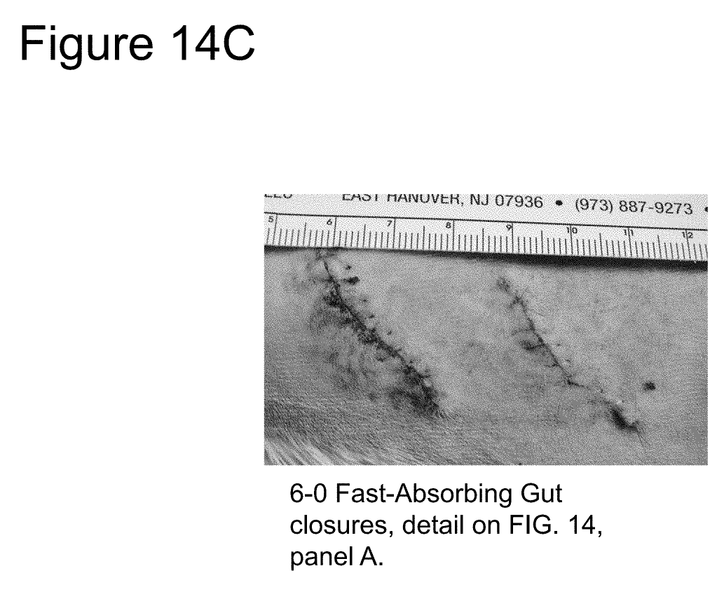

Sprague Dawley rats were anesthetized by dosing intraperitoneally with an anesthetic cocktail including 50 mg/kg of ketamine and 4 mg/kg of xylazine. As necessary, inhaled isoflurane was used to adjust anesthesia during the procedure. A plurality of three centimeter incision wounds were made in dorsal skin of the rats using a surgical scalpel. The incisions were ligated using either fibrin microthread suture, a 6-0 ETHILON® suture, or a 6-0 fast-absorbing gut suture using a simple interrupted transcutaneous suture pattern. Test animals were harvested at 4, 7, 14, and 28 days post-operatively. FIGS. 14A-C show dorsal skin incisions closed by both fibrin microthread suture and fast-absorbing gut suture. FIG. 14A shows a contralateral repair with both fibrin microthread suture and fast-absorbing gut suture while FIGS. 14B and 14C show detail on the fibrin microthread and fast-absorbing gut sutures respectively.

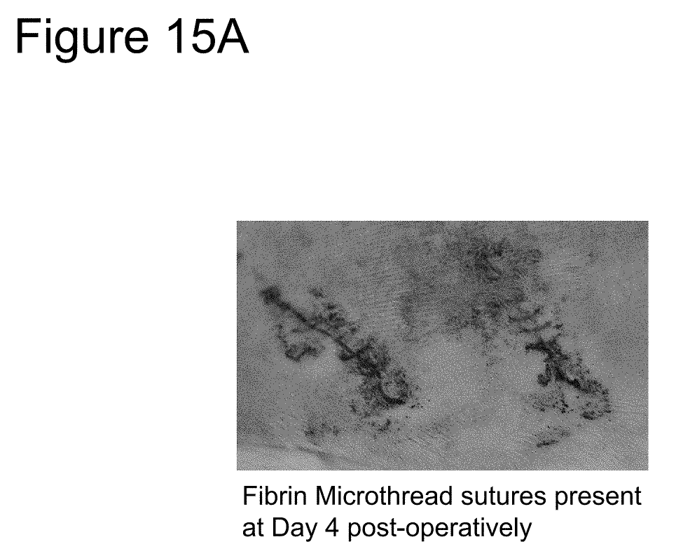

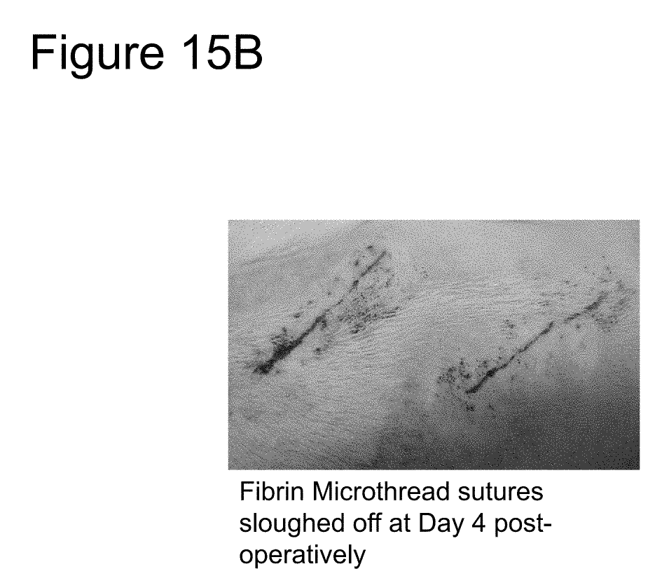

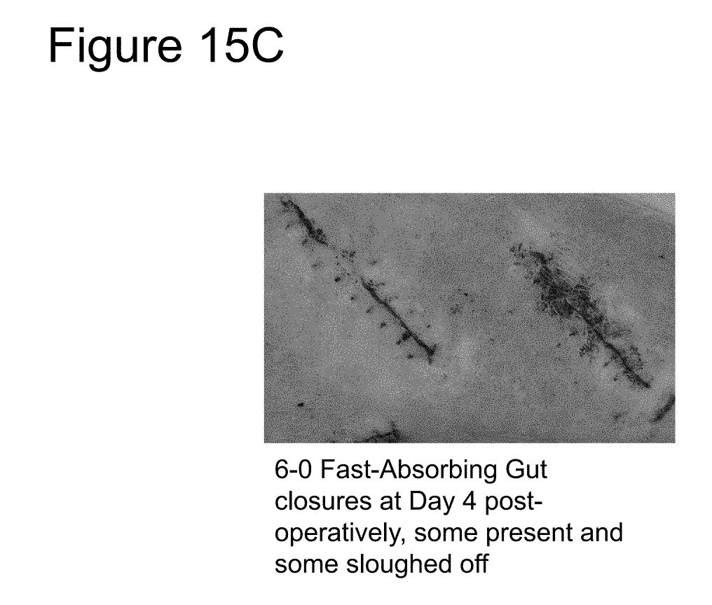

FIGS. 15A-C show fibrin microthread suture and fast-absorbing gut suture repairs at 4 days post-operatively. FIGS. 15A and B show both presence and absence of fibrin microthread at this timepoint while the wounds remain closed. This behavior is consistent with what would be desired in a typical post-operative recovery from skin closure with suture absorbing as native healing continues to strengthen the wound bed. FIG. 15C shows fast-absorbing gut suture both present and absent along the length of two healing incisions at the 4 day timepoint suggesting comparable duration of tissue reinforcement provided by fast-absorbing gut and fibrin microthread suture.

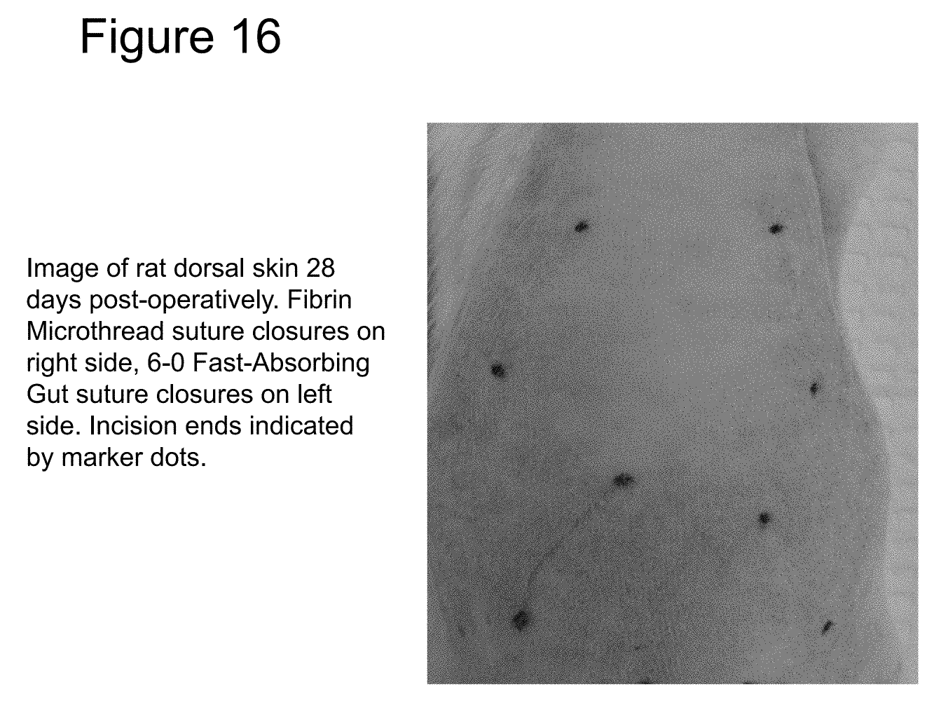

FIG. 16 shows dorsal skin of a rat at post-operative day 28 having had a series of 3 cm incisions repaired by fibrin microthread suture (right side) and 6-0 fast-absorbing gut suture (left side). Due to the extent and quality of healing, closed incision sites have been indicated by pen marks at their endpoints. Of interest, there appears to be greater residual scarring at the sites closed by 6-0 fast-absorbing gut suture.

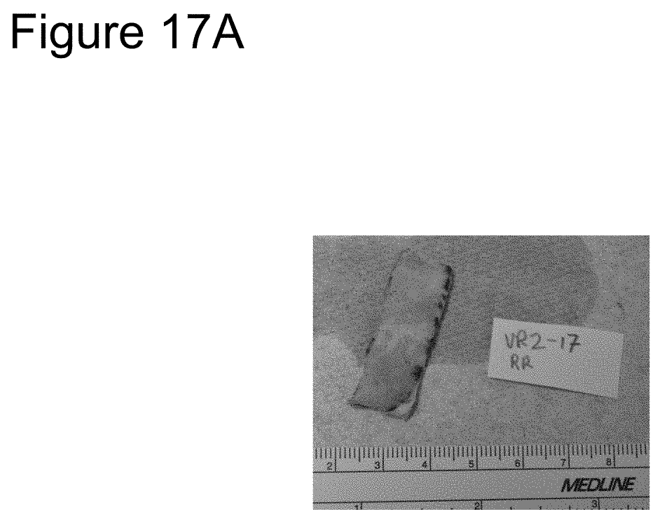

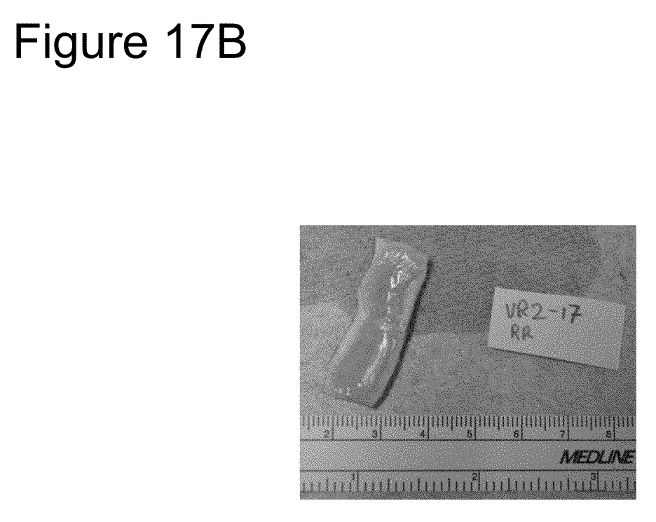

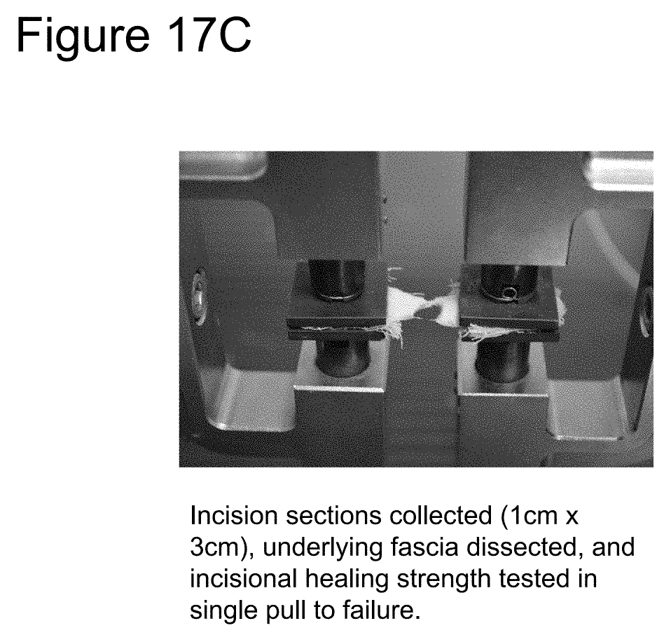

FIGS. 17A-C illustrate the tensile testing specimen preparation and testing. Briefly, representative tissue tags were collected as a 3 cm×1 cm tag oriented perpendicularly to the direction of the initial incision and centered about the midpoint of the incision. This was done in order to test the relative strength of the healing tissue following incision and closure. Samples were loaded onto an ElectroPuls E-1000 unit from Instron (Canton, Mass.) equipped with a calibrated 50 N load cell. After loading in negligible tension with a gauge length of 1 cm, samples were pulled at a strain rate of 100% per minute until failure. Maximum load, displacement at failure, and device cross-sectional area were recorded. A minimum of n=6 mechanical samples were processed from each incision closure type per each timepoint.

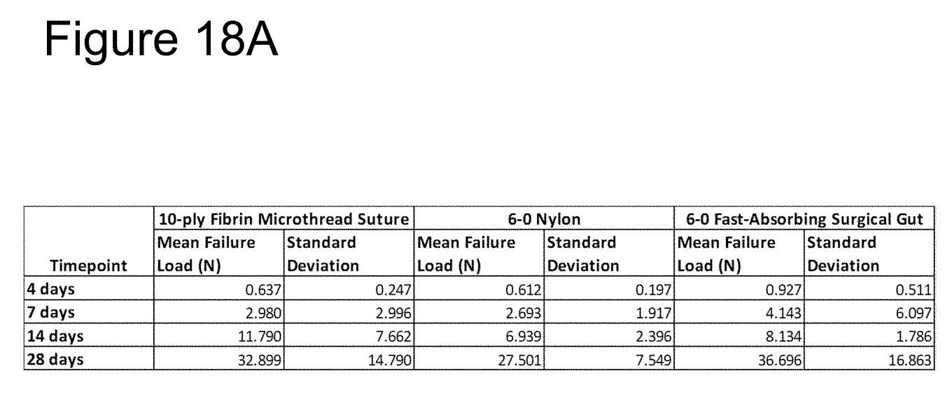

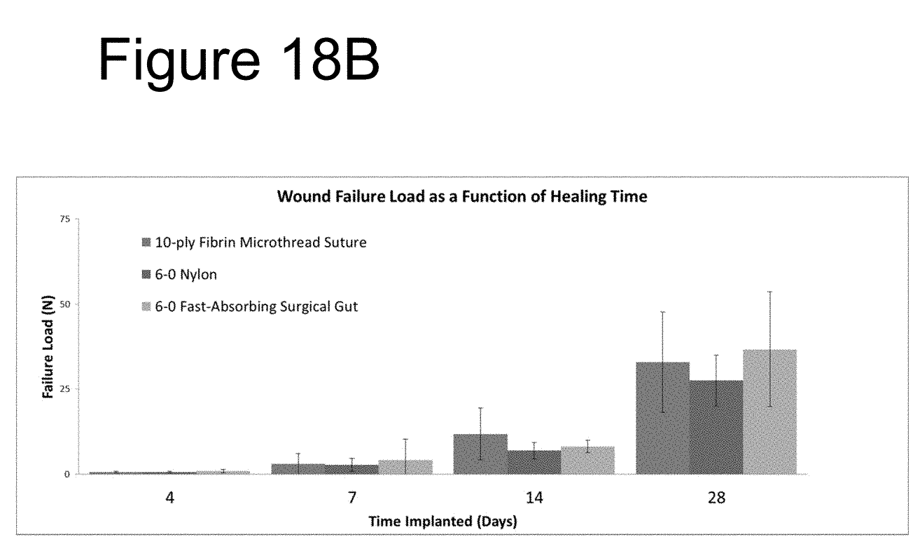

FIGS. 18A-B shows the resulting mean maximum loads as developed in the tensile testing performed on incisions sites described previously. No significant differences were observed between any of the sample groups at any of the given timepoints. This data demonstrates that wound healing strength was comparable for fibrin microthread sutures as compared to conventional suture materials such as the nylon of 6-0 ETHILON® suture and the collagen of 6-0 fast-absorbing gut suture

### Example 5

### Production of Fibrin Microthreads

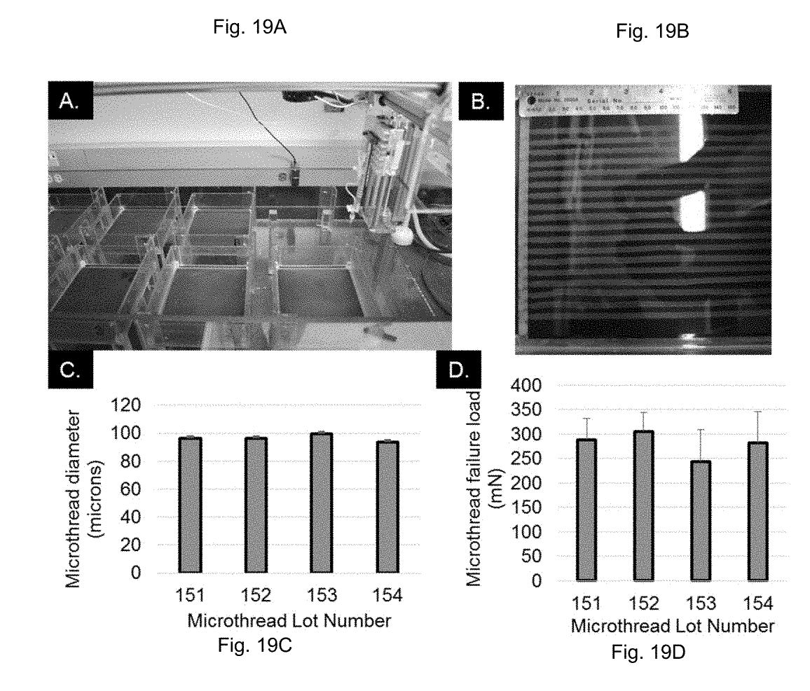

Fibrin microthreads are produced using a three axis electromechanical extrusion head to “print” fibrin microthreads in a buffer bath (see FIGS. 19A-19B). The production system allows for the generation of threads with lot-to-lot consistency in both thread diameter and thread failure load (see FIGS. 19C and 19D). The produced fibrin microthreads are used as building blocks for additional products such as sutures.

### Example 6

### Production of Fibrin Microthread Sutures

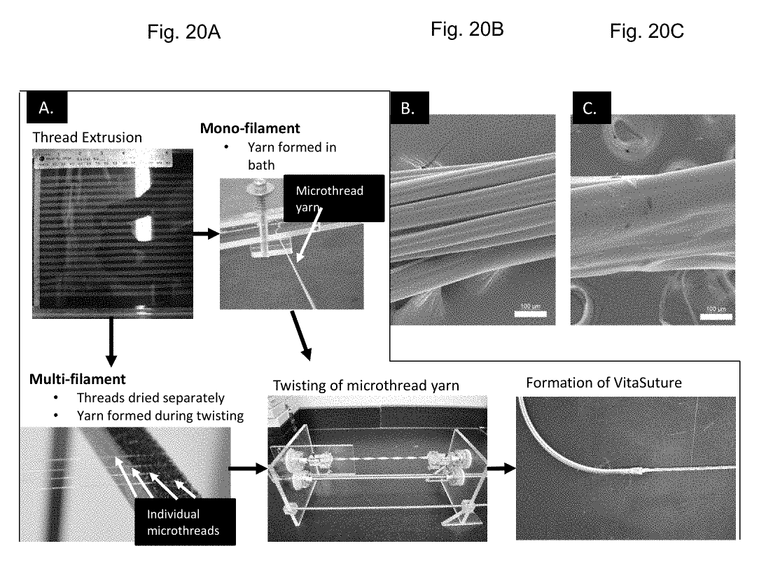

Suture products can be generated in both a multifilament and monofilament form of varying sizes. Multifilament sutures are formed by fully drying individual microthreads after formation in the bath. Varying numbers of dried threads can then be twisted together with a controlled twist density to produce a multifilament microthread yarn. FIGS. 20A-20C differentiate the production process for multi- and mono-filament sutures and show scanning electron micrographs of each type of suture.

Monofilament sutures are produced from the same microthread extrusion process as used for multifilament sutures. Prior to removal from the extrusion bath, varying numbers of microthreads are carefully pulled together in the buffer solution to form a single, cohesive thread. The thread is then removed from the bath and allowed to dry.

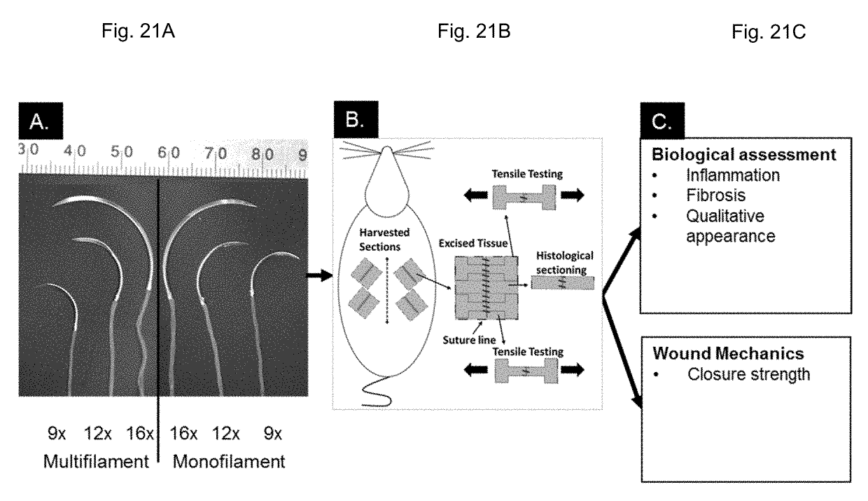

After drying of both the multi- and mono-filaments, sutures are formed by inserting the dried material into the bore hole of a standard drilled-end surgical needle and crimping the suture in place. Use of this process allows for sutures to be readily attached to a variety of needle types for different purposes. See FIGS. 21A-21C. Sutures are then packaged and sterilized via a standard 12-hour ethylene oxide cycle for use in the sterile operating room.

### Example 7

### Variation of Fibrin Microthread Mechanics

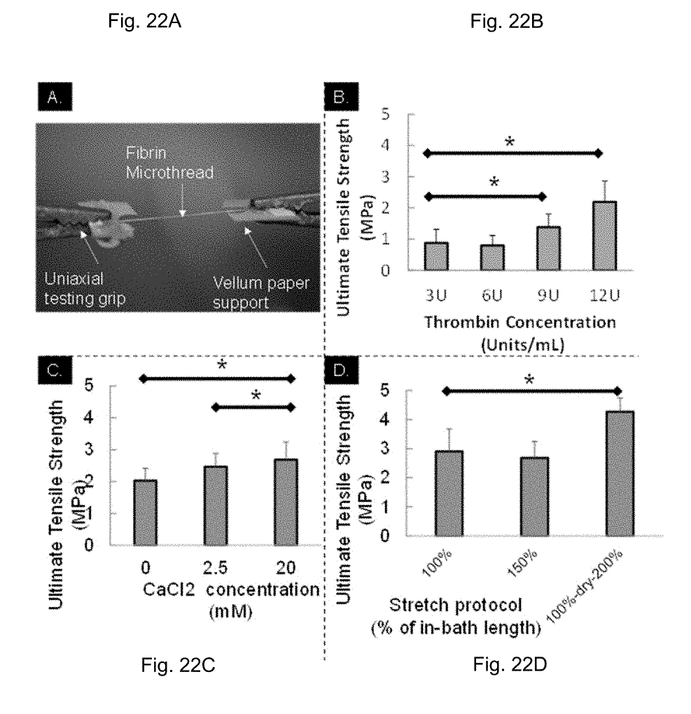

Methods of manufacturing fibrin microthreads as described herein involve the use of an automated system which produces microthreads of consistent diameter and mechanical strength. To further increase the mechanical strength of the microthreads, various parameters of the microthread extrusion process were modified and tested. Specifically, the fibrin microthreads were assessed mechanically. After formation, dry threads were glued to a piece of vellum paper using silicone glue. Microthreads were rehydrated for at least 10 minutes in lactated ringer's solution and thread diameters were measured using a light microscope. Threads were then tested uniaxially to failure at 200% strain/min on an Instron Electropuls E1000 tensile testing device with a 1 Newton load cell (see FIG. 22A). Results are reported as ultimate tensile strength (MPa) using initial thread diameter and assuming circular cross-section to determine area.

Some of the primary parameters that affect the physical and mechanical properties of fibrin microthreads include the bath composition, the extrusion head velocity, the thrombin and fibrinogen concentrations and the extrusion flow rate. Table 2 below lists the parameters used in an initial microthread extrusion process as well as an improved process with modified parameters for increasing the mechanical strength of the fibrin microthreads used to form sutures (“After” column).

TABLE 2

| Extrusion | | |
|---|---|---|
| Parameter | Before | After |
| Bath | 10 mM HEPES | 10 mM HEPES + 20 mM CaCl₂ |
| Composition | | |
| Extrusion flow | 0.39 mL/min | 0.39 mL/min |
| rate | | |
| Fibrinogen | 4.9% Clottable/mL | 4.9% Clottable/mL |
| concentration | | |
| Thrombin | 30 Units/mL | 60 Units/mL |
| concentration | | |
| Stretching | 100% out of bath | 100%-rehydrate-200% |
| protocol | | |

One of the varied parameters is thrombin concentration. Specifically, threads were produced using the standard extrusion parameters in the “Before” column in Table 2 except varying thrombin concentration. Samples were processed with 3, 6, 9, and 12 U/mL of thrombin, respectively, while maintaining all other parameters constant. Samples were then tested uniaxially to failure. As shown in FIG. 22B, increasing thrombin concentration improved fibrin microthread strength. Further, increasing strength of the fibrin microthreads resulted in an increase in ultimate tensile strength (UTS) compared to initial levels. In particular, a thrombin concentration of 12 units/mL resulted in significant increases in UTS compared to other concentrations.

Another varied parameter is the addition of CaCl₂ to the buffer bath. Calcium chloride (CaCl₂) may play an important role in fibrinogen polymerization. Accordingly, fibrin microthreads were extruded with different concentrations of CaCl₂ in the extrusion bath. Concentrations tested were: 0, 2.5 and 20 mM. After thread formation, threads were tested mechanically. Addition of calcium chloride to the extrusion bath increased thread tensile strength. As shown in FIG. 22C, microthread UTS was significantly increased compared to 0 and low (2.5 mM) concentration of CaCl₂ when CaCl₂ concentration in the bath was increased to 20 mM.

An additional step of pre-stretching was included into microthread processing. To enhance alignment of fibrin during formation, the initial fibrin microthread production process maintained threads at 100% of their initial length during the drying step of the process. In a first group of this study, a standard 100% stretch protocol was used. In a second group of this study, threads were stretched to 150% of their initial length immediately out of the bath. In the third group, threads were maintained at 100% in bath length and allowed to dry. The threads were then rehydrated in DI water and stretched to 200% in bath length. It was discovered that drying of threads prior to stretching improved thread mechanical strength. Microthread UTS was further increased with the addition of a stretching and drying step to the manufacturing process. The process of drying the threads at 100% in bath length, rehydrating and stretching to 200% in-bath length resulted in a significant (p<0.05) increase in thread mechanical strength compared to both 100% in-bath length and 150% in-bath length protocols (see FIG. 22D). Accordingly, a set of key process variables have been identified which may be used to manipulate the mechanical properties of fibrin microthreads. By modulating one or more of these variables, the ultimate tensile strength of the fibrin microthreads are increased. These experimental procedures demonstrate that the mechanical characteristics of fibrin microthreads can be improved without employing cross-linking agents or harsh reaction conditions.

### Example 8

### Monofilament Thread Assembly

Aqueous fibrinogen solution and a thrombin are co-extruded using the previously described microthread extrusion equipment in a saline bath containing standard HEPES buffer and CaCl₂. Concentration ranges previously described remain appropriate. The machine is programmed in such a fashion that it traces a similar pattern in the bath such that multiple layers of fibrin are deposited. Typical time between layer depositions over a given point are approximately 15 seconds. Conditions have also been evaluated using longer periods of time, for example, approximately 3 minutes. Either condition forms a monofilament fibrin thread.

Methods as described herein can produce a single layer of deposition. Methods as described herein can also produce a 12-layer deposition to form a monofilament fibrin thread which may be used in suture applications. Monofilament fibrin threads formed from 3, 6, 9, 10, 12, and 14-layer depositions have also been produced.

These monofilament threads post-extrusion may be stretched mechanically in order to increase their ultimate tensile strength either prior to bath removal (in-bath) or following bath removal. For example, the monofilament thread may be stretched in-bath for 6-8 minutes following initiation of extrusion on a particular point.

If stretching, it is desirable to stretch these threads within 30 or more minutes of initiating extrusion or under 2 minutes. Threads may also be stretched within 10-30 minutes of initiating extrusion or 2-6 minutes. In addition, threads may also be stretched within 6-8 minutes of initiating extrusion. The amount of time for stretching varies depending on ease of handling the materials and their resultant mechanical properties.

Stretching may be varied between 100-250%. For example, the monofilament thread may be stretched to 200%.

Following a stretching step, resultant monofilament materials may be twisted to improve overall cross-sectional uniformity. Twisting may be performed to a level of between 0.1 twists/cm to around 3 twists/cm. For example, twisting may be performed at 1 twist/cm prior to drying.

Following stretching (with or without twisting), the resultant fibrin materials are dried under ambient conditions. For example, drying may take approximately 6 hours.

Following drying, a bored-end needle may be attached to the fibrin monofilament via a standard crimping mechanism to generate a suture. Alternatively, the threads may be passed through the eyelet of an eyelet-style needle, then rehydrated in a saline buffer and hereby attached and appropriately organized. For example, bored-end needles or eyelet-style needles may be crimped on at this stage.

In the case where an eyelet-style needle is used, additional twisting may be applied post-needle attachment to further organize the suture yarn. Twisting may be performed to a level of between 0.1 twists/cm to around 3 twists/cm. In certain instances, twisting may be performed in a direction counter to any initial twisting applied to the yarn, generating a balanced yarn.

Provided below in Table 3 is a characterization data of a 10-pass fibrin monofilament thread stretched 6-8 minutes post-extrusion.

Threads were subjected to single-pull-to-failure testing using an Instron 3342 test system with a 50N load cell and pneumatic yarn grips. A gauge length of 55 mm was used and a strain rate of 200% per minute. Maximum load and extension at failure were both recorded. Prior to testing, sample diameters were measured in order to calculate ultimate tensile strength.

Dry diameter is recorded using a Willrich 65-0642Q digital yarn micrometer featuring an AQD-2600N digital indicator according to USP 29.861. Briefly, for dry diameter measurement a monofilament was placed under ambient conditions between the platens of the yarn micrometer under a compressive load of 210 g spread over the platen area, a circular foot 12.7 mm in diameter. Hydrated diameter measurement was performed in the same fashion as dry diameter measurement with the exception of the fact that fibrin monofilaments were first incubated for a minimum of 10 and a maximum of 30 minutes in and excess of lactated Ringer's solution.

TABLE 3
Representative Physical Properties for a 10× Layered Fibrin Monofilament

| | | | | | | | | Ultimate Tensile | | | |
|---|---|---|---|---|---|---|---|---|---|---|---|
| | | | | | | | | Strength | | Young's Modulus | |
| Maximum Load | | Maximum Extension | | Dry Diameter | | Hydrated Diameter | | (N/mm2 based on | | (Mpa based on | |
| (N) | | (mm) | | (microns) | | (microns) | | hydrated diameter) | | hydrated diameter) | |
| | Standard | | Standard | | Standard | | Standard | | Standard | | Standard |
| Mean | Deviation | Mean | Deviation | Mean | Deviation | Mean | Deviation | Mean | Deviation | Mean | Deviation |
| 2.011 | 0.426 | 92.535 | 13.153 | 443.755 | 42.479 | 334.000 | 18.660 | 22.721 | 3.792 | 8.536 | 1.545 |

### EQUIVALENTS

While various embodiments of the system, methods and devices have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and such modification are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method for promoting wound closure in a host tissue, comprising applying a fibrin microthread suture to the host tissue of a subject in need thereof.

2. The method of claim 1, wherein the fibrin microthread suture is associated with one or more of a substrate or a braided yarn or other hierarchically organized rope, a woven or non-woven mesh, a surgical needle, a surgical pin, a surgical screw, a surgical plate, a physiologically acceptable patch, a dressing, a bandage, or a natural or mechanical valve.

3. The method of claim 1 or 2, wherein the fibrin microthread suture has a histopathological score of extent of resorption in the host tissue of greater than about 1.

4. The method of claim 3, wherein the fibrin microthread suture has a histopathological score of extent of resorption in the host tissue of less than about 3.

5. The method of any one of claims 1-3, wherein the fibrin microthread suture is absorbed in the host tissue in about 3 to about 28 days.

6. The method of claim 5, wherein the fibrin microthread suture is absorbed in the host tissue in about 4 to about 10 days.

7. The method of claim 5, wherein the fibrin microthread suture is absorbed in the host tissue in about 7 to about 14 days.

8. The method of claim 5, wherein the fibrin microthread suture is absorbed in the host tissue in about 14 to about 28 days.

9. The method of any one of claims 1-8, wherein the method prevents or reduces inflammation at the host tissue or proximal to the fibrin microthread suture.

10. The method of claim 9, wherein the fibrin microthread suture has a histopathological score of overall inflammation in the host tissue of less than about 1.5.

11. The method of claim 10, wherein the fibrin microthread suture has a histopathological score of overall inflammation in the host tissue of less than about 1.3.

12. The method of any one of claims 1-11, wherein the method prevents or reduces collagen deposition at the host tissue or proximal to the fibrin microthread suture.

13. The method of claim 12, wherein the fibrin microthread suture has a histopathological score of collagen deposition in the host tissue of less than about 1.5.

14. The method of claim 13, wherein the fibrin microthread suture has a histopathological score of collagen deposition in the host tissue of less than about 1.3.

15. The method of any one of claim 1-14, wherein the method prevents or reduces scarring at the host tissue or proximal to the fibrin microthread suture.

16. The method of any one of claims 1-15, wherein the method prevents or reduces cellular infiltration or proliferation at the host tissue or proximal to the fibrin microthread suture.

17. The method of any one of claims 1-16, wherein the host tissue is a soft tissue.

18. The method of claim 17, wherein the soft tissue is selected from simple squamous epithelia, stratified squamous epithelia, cuboidal epithelia, or columnar epithelia, connective tissue, and muscle tissue.

19. The method of claim 17, wherein the soft tissue is skin.

20. The method of any one of claims 1-19, wherein the wound is a surgical wound.

21. The method of claim 20, wherein the surgical wound results from cosmetic surgery.

22. The method of claim 21, wherein the cosmetic surgery is selected from blepharoplasty, rhinoplasty, rhytidectomy, chin augmentation, facial implants, ear surgery, hair implantation, cleft lip and cleft palate repair, abdominoplasty, arm lift, thigh lift, breast reduction, breast augmentation, body contouring, liposuction, and hand surgery.

23. The method of claim 20, wherein the surgical wound results from cardiac surgery, skeletal muscle repair, congenital or incision hernia repair, abdominal surgery, laproscopic incision closure, organ prolapse surgery, gastrointestinal surgery, neurosurgery, severed limb reattachment surgery, pulmonary surgery, hepatic surgery, renal surgery, ocular surgery, periodontal surgery, and orthopedic surgery.

24. The method of any one of claims 1-23, wherein the fibrin microthread suture comprises an additional therapeutic agent.

25. The method of claim 24, wherein the additional therapeutic agent is one or more of a growth factor, a protein, a chemotherapeutic agent, a vitamin, a mineral, an antimicrobial agent, a small organic molecule, and a biological cell.

26. A method for promoting wound closure in a host tissue, comprising applying a fibrin microthread suture to the host tissue of a subject in need thereof, wherein the fibrin microthread suture provides one or more of:
(i) faster absorption in the host tissue;
(ii) reduced inflammation in the host tissue;
(iii) reduced collagen deposition in the host tissue; and
(iv) reduced scarring in the host tissue,
relative to a conventional suture.

27. The method of claim 26, wherein the conventional suture is polyglactin (VICRYL®) or surgical gut.

28. The method of any one or claim 26 or 27, wherein the host tissue is soft tissue, optionally selected from simple squamous epithelia, stratified squamous epithelia, cuboidal epithelia, or columnar epithelia, connective tissue, and muscle tissue

29. The method of any one or claims 26-28, wherein the wound is associated with a cosmetic surgery, optionally selected from blepharoplasty, rhinoplasty, rhytidectomy, chin augmentation, facial implants, ear surgery, hair implantation, cleft lip and cleft palate repair, abdominoplasty, arm lift, thigh lift, breast reduction, breast augmentation, body contouring, liposuction, and hand surgery.

30. The method of any one or claims 26-29, wherein the fibrin microthread suture comprises an additional therapeutic agent, optionally selected from one or more of a growth factor, a protein, a chemotherapeutic agent, a vitamin, a mineral, an antimicrobial agent, a small organic molecule, and a biological cell.

31. A method for promoting wound closure in a host tissue, comprising applying a fibrin microthread suture to the host tissue of a subject in need thereof, wherein the subject has not received a conventional suture.

32. The method of claim 31, wherein the conventional suture is polyglactin (VICRYL®) or surgical gut.

33. The method of any one or claim 31 or 32, wherein the tissue is soft tissue, optionally selected from simple squamous epithelia, stratified squamous epithelia, cuboidal epithelia, or columnar epithelia, connective tissue, and muscle tissue

34. The method of any one or claims 31-33, wherein the wound is associated with a cosmetic surgery, optionally selected from blepharoplasty, rhinoplasty, rhytidectomy, chin augmentation, facial implants, ear surgery, hair implantation, cleft lip and cleft palate repair, abdominoplasty, arm lift, thigh lift, breast reduction, breast augmentation, body contouring, liposuction, and hand surgery.

35. The method of any one or claims 31-34, wherein the fibrin microthread suture comprises an additional therapeutic agent, optionally selected from one or more of a growth factor, a protein, a chemotherapeutic agent, a vitamin, a mineral, an antimicrobial agent, a small organic molecule, and a biological cell.

36. The method of any one of claims 1-35, wherein the fibrin microthread is a multifilament fibrin microthread.

37. The method of any one of claims 1-35, wherein the fibrin microthread is a monofilament fibrin microthread.

38. The method of any one of clams 1-37, wherein the fibrin microthread is produced by a method comprising the steps of:
(a) combining a first volume of fibrinogen with a second volume of a molecule capable of forming fibrin from fibrinogen to form a mixture;
(b) transferring the mixture to a lumen containing device;
(c) diposing the distal end of the lumen containing device into an aqueous bath; and
(d) extruding the mixture from the distal end of the lumen containing device thereby forming the fibrin microthread in the aqueous bath.

39. The method of claim 38, further comprising the step of removing the fibrin microthread from the aqueous bath.

40. The method of claim 39, further comprising the step of mechanically stretching the fibrin microthread after removal from the aqueous bath.

41. A method for producing a monofilament fibrin microthread, comprising the steps of:
a) extruding fibrin microthreads into a bath and forming a yarn of microthreads in the bath;
b) stretching the yarn of microthreads to about 200% of initial length; and
c) drying the stretched the yarn of microthreads to produce a monofilament fibrin microthread.

42. A method for producing a monofilament fibrin microthread, comprising the steps of:
a) extruding fibrin microthreads into a bath and forming a yarn of microthreads in the bath;
b) drying the yarn of microthreads;
c) rehydrating the yarn of microthreads;
d) stretching the yarn of microthreads to about 200% of initial length; and
e) drying the stretched the yarn of microthreads to produce a monofilament fibrin microthread.

43. The method of claim 41, comprising the additional steps of:
f) rehydrating the monofilament fibrin microthread; and
g) attaching the monofilament fibrin microthread to a needle

44. The method of any one of claims 41-43, wherein the bath comprises about 10 mM HEPES and about 20 mM CaCl₂.

45. The method of any one of claims 41-44, wherein the extrusion is at a flow rate of about 0.3 ml/min, or about 0.35 mL/min, or about 0.40 mL/min, or about 0.45 mL/min, or about 0.50 mL/min.

46. The method of any one of claims 41-45, wherein the monofilament fibrin microthread is less susceptible to infection when used in a suture as compared to a multifilament fibrin microthread.

47. The method of any one of claims 41-46, wherein the method does not comprise the use of a cross-linking agent.

48. The method of any one of claims 41-47, wherein in step a) the fibrin microthreads are extruded into the bath in layers.
